SEQUENCE LISTING (5 sequences)

SEQ ID NO: 1 - DNA, 782 bp, Homo sapiens
gggctccctgcctcgggctctcaccctcctctcctgcagctccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccaggccattcgcaccagccaccacccactcccaccccctgtagtgctcccacccctggactggtggcccccaccctgcgggaggcctccccatgtgcctgcgccaagagacagacagagaaggctgcaggagtcctttgttgctcagcagggcgctctgccctccctccttccttcttgcttctaatagccctggtacatggtacacacccccccacctcctgcaattaaacagtagcatcgcctccctctgaaaaaaaaaaaaaaaaaaaaaaa

SEQ ID NO: 2 - DNA, 20 bp, Artificial sequence
  primer
taggataatcccgggtggca

SEQ ID NO: 3 - DNA, 21 bp, Artificial sequence
  primer
gtctgttgcctggctcttagt

SEQ ID NO: 4 - DNA, 22 bp, Artificial sequence
  primer
ctcctcctgttcgacagtcagc

SEQ ID NO: 5 - DNA, 21 bp, Artificial sequence
  primer
cccaatacgaccaaatccgtt

(19) United States
(12) Patent Application Publication
(10) Pub. No.: US 2022/0220557 A1
(43) Pub. Date: Jul. 14, 2022

(54) Use of Cystatin SN in Detecting Chronic Rhinosinusitis with Nasal Polyps Subtype and Predicting Sensitivity of Patient to Glucocorticoid

(71) Applicants: BEIJING TONGREN HOSPITAL, CAPITAL MEDICAL UNIVERSITY, Beijing (CN); BEIJING INSTITUTE OF OTOLARYNGOLOGY, Beijing (CN)

(72) Inventors: Luo Zhang, Beijing (CN); Chengshuo Wang, Beijing (CN); Bing Yan, Beijing (CN)

(21) Appl. No.: 17/142,228

(22) PCT Filed: Jun. 27, 2019
(86) PCT No.: PCT/CN2019/093286

§ 371 Date: Jan. 5, 2021

(30) Foreign Application Priority Data
Jul. 3, 2018 (CN) 201810717432.2
Jul. 3, 2018 (CN) 201810720279.9
Jun. 17, 2019 (CN) 201910521050.7

(51) Int. Cl. C12Q 1/6883; C12Q 1/6851; C12Q 1/6806

(52) U.S. Cl. CPC C12Q 1/6883; C12Q 1/6851; C12Q 2600/156; C12Q 2600/166; C12Q 2600/112; C12Q 1/6806

(57) ABSTRACT

Provided are a kit for detecting chronic rhinosinusitis with nasal polyps subtype, and the use of a CST1 gene as a biomarker and a method for detecting chronic rhinosinusitis with nasal polyps subtype. The CST1 gene, as a biomarker, can detect chronic rhinosinusitis with nasal polyps subtype or predict the sensitivity of a patient with chronic rhinosinusitis with nasal polyps to glucocorticoid. The detection method includes PCR using specific primers of the CST1 gene, such as real-time fluorescence quantitative PCR, so as to detect the expression amount of the CST1 gene.

### CROSS-REFERENCE TO RELATED APPLICATIONS

The present disclosure claims the priority to the Chinese patent application with the filing No. 201810717432.2 filed with the Chinese Patent Office on Jul. 3, 2018 and entitled “Kit for Detecting Chronic Rhinosinusitis with Nasal Polyps Subtype, and Application of CST1 Gene as Biomarker”, the Chinese patent application with the filing No. 201810720279.9 filed with the Chinese Patent Office on Jul. 3, 2018 and entitled “Method for Detecting CST1 Gene Expression Level in Nasal brushing cells and Application of Method”, and the Chinese patent application with the filing No. 201910521050.7 filed with the Chinese Patent Office on Jun. 17, 2019 and entitled “Application of Cystatin SN in predicting Sensitivity of Chronic Rhinosinusitis with Nasal Polyps Patient on Glucocorticoid”, the contents of each are incorporated herein by reference in entirety.

### TECHNICAL FIELD

The present disclosure belongs to the field of biomedical technologies, and in particular, to a kit for detecting chronic rhinosinusitis with nasal polyps subtype and application of a CST1 gene as a biomarker, a method for detecting an expression level of a CST1 gene in nasal brushing cells, and application of Cystatin SN in predicting sensitivity of a patient with chronic rhinosinusitis with nasal polyps to glucocorticoid.

### BACKGROUND ART

Chronic rhinosinusitis with nasal polyps (CRSwNP) is chronic inflammation of nasal sinus mucosa, and the formation of polyps in the nasal cavity or middle nasal meatus may be seen in physical examination. Common symptoms of CRSwNP are nasal obstruction, rhinorrhea or backflow of nasal discharge, hyposmia, facial stuffiness or pressure for more than 12 weeks. The morbidity is about 0.5˜4%. CRSwNP is often accompanied by asthma and allergic rhinitis, and it has been reported that 7% of asthma patients suffer from CRSwNP, and 26˜48% of CRSwNP patients suffer from asthma. The pathogenesis of CRSwNP has not been determined at present, and mucosal epithelial cell destruction, host immune system imbalance and pathogenic microorganism invasion may be main causes of CRSwNP. The main treatment for CRSwNP is surgery and medication. However, researches show that even with standardized medication or surgical treatment, the recurrence rate of chronic rhinosinusitis with nasal polyps is still as high as 56%, which seriously affects the patients' life quality, and meanwhile brings high medical expenses, but curative treatment methods are lacked clinically, therefore, CRSwNP has become the focus of the field of nasal science researches.

The CRSwNP can be divided into eosinophilic CRSwNP (ECRSwNP) and noneosinophilic CRSwNP (nonECRSwNP) according to the degree of eosinophil infiltration. The clinical manifestations, drugs and prognosis of the two are different. The clinical symptoms of eosinophilic CRSwNP are heavier, primarily nasal obstruction and hyposmia, mostly combined with asthma, and with a higher postoperative recurrent rate. The degree of eosinophil infiltration in nasal polyp tissues is most closely related to relapse, and when the percentage of the eosinophils in the tissues exceeds 27%, the relapse risk will exceed 90%. The sensitivity of eosinophilic nasal polyp to glucocorticoid drugs is significantly higher than that of the noneosinophilic CRSwNP. The clinical symptoms of the noneosinophilic CRSwNP are generally milder, with smaller probability of being combined with asthma, milder airway inflammation, lower postoperative recurrence rate than that of the eosinophilic CRSwNP, and good therapeutic response to macrolide drugs. The eosinophilic CRSwNP predominates in the western countries, mainly manifesting TH2 inflammatory response, while the eosinophilic CRSwNP and the noneosinophilic CRSwNP both account for about half in China, and the noneosinophilic CRSwNP mainly manifests TH1/TH17 inflammatory response. In summary, the eosinophilic CRSwNP and the noneosinophilic CRSwNP differ significantly in immunopathological type, clinical symptom, medication response and prognosis. Different inflammation/pathological types of chronic rhinosinusitis with nasal polyps have different treatment strategies. Hence, identification of pathological type of chronic rhinosinusitis with nasal polyps is particularly important.

Currently, two subtypes are judged mainly based on tissue pathological specimen staining after nasal mucosal biopsy, which lacks noninvasive biological markers for discrimination diagnosis. The polyp specimens of the patient, after being obtained under nasal endoscope, are subjected to routine treatment on pathological specimens, such as paraffin fixation, then staining with hematoxylin and eosin, next, the number of inflammatory cells (main inflammatory cells include eosinophils, neutrophils, lymphocytes, plasma cells) infiltrating tissues are observed through a high-power microscope, to perform cell typing. The disadvantages of nasal mucosal pathological biopsy are as follows: 1. it is invasive examination: the patients' infection risk is increased, and it is not applicable to lower-immunity human groups such as children and the elderly; nasal bleeding during sampling often causes fear and worry of the patients; 2. it is difficult to obtain real-time dynamic change information of the disease: no pathological biopsy can be performed on the healing phase mucosa after surgery, the clinical data, however, shows that the pathological classification of chronic rhinosinusitis with nasal polyps will be regressed with medication, surgical treatment, etc., then the pathological biopsy result before treatment cannot represent the characteristics of all periods of the disease; 3. it is time-consuming and increases medical costs, because it generally takes 3˜4 working days from obtaining tissue samples to obtaining pathological results, then as the results cannot be obtained on the day or the next day, extra transportation expenses, lodging fees, and registration fees are incurred to patients seeking medical service in other places, and the medical costs are increased; 4. there are certain human errors, as the number of inflammatory cells counted by different pathologists may be different, which affects the judgment of the polyp type; 5. the tissue pathological section is relatively restricted, and can only reflect the inflammatory state of the specimen at the position of the section, but cannot reflect a full view of the tissues, which may cause misdiagnosis; and 6. each slide needs a pathologist to manually count, and it is difficult to operate in batches.

It can thus be seen that it is a technical difficulty to be solved by a person skilled in the art to provide a method capable of quickly and accurately detecting a chronic rhinosinusitis with nasal polyps subtype in batches without relying on pathological biopsy of nasal mucous.

The real-time quantitative PCR (real-time fluorescence quantitative PCR) emerging in recent years compensates for the disadvantages of the above technology. In this method fluorescent groups are added to a PCR reaction system, the whole PCR process is monitored in real time through accumulation of fluorescent signals, and finally the measured DNA samples are subjected to quantitative analysis according to the fluorescent signals. This method is easy to operate, and has high sensitivity, good repeatability and high accuracy of results. However, the prior art does not disclose a method for effectively detecting the expression level of the CST1 gene in nasal brushing cells so as to better acquire the situation of content of the CST1 gene in the nasal brushing cells.

The chronic rhinosinusitis with nasal polyps manifests the symptoms such as nasal obstruction, rhinorrhea, sneezing, and itching. The intrinsic cause is increased inflammatory factors. It is generally believed that TH1/TH2/TH17 factor imbalance is its immunological cause. This cause, in turn, is closely related to the response degree of the disease to clinical drugs. Generally, TH2 type inflammatory response has relatively good response to glucocorticoid, while the response degree of the TH1/TH17 type factors is relatively poor.

As to clinical practice, however, how to judge the immunity type is still a difficult point in diagnosis and treatment. The conventional gold standard employs a manner of pathological detection, i.e. disease tissues are obtained through biopsy, and undergo paraffin embedding, sectioning, hematoxylin and eosin staining, and counting to obtain numerical values of various inflammatory cells, and subsequently to judge the inflammation type after calculating the percentage. However, to obtain the tissues through biopsy increases the possibility of bleeding and infection of patients. Therefore, in clinical practice, glucocorticoid is usually administered to patients having no glucocorticoid contraindications, and then it is judged whether the glucocorticoid is effective according to the endoscopic result so as to guide subsequent medication. Although the use of short-term or topical glucocorticoid hardly brings about systemic side effects of hormone, increased diagnostics costs are still caused to patients who are insensitive to glucocorticoid.

### SUMMARY

The present disclosure proposes a kit for detecting chronic rhinosinusitis with nasal polyps subtype and application of a CST1 gene as a biomarker.

The present disclosure provides a kit for detecting chronic rhinosinusitis with nasal polyps subtype, wherein the kit includes a specific primer of a CST1 gene.

The present disclosure provides use of a CST1 gene as a biomarker in preparation of a product for detecting chronic rhinosinusitis with nasal polyps subtype.

The present disclosure provides a method for detecting an expression level of a CST1 gene in nasal brushing cells, including steps of: extracting RNA from nasal brushing cells, making total RNA to undergo reverse transcription to cDNA, performing real-time quantitative PCR amplification on a CST1 gene and a reference gene in the cDNA by using a specific primer of the CST1 gene and a specific primer of the reference gene, respectively, by adopting quantitative polymerase chain reaction, and calculating the expression level of the CST1 gene based on a detection result of an amplification product.

Use of the above method for detecting an expression level of a CST1 gene in nasal brushing cells in preparation of a kit for detecting chronic rhinosinusitis with nasal polyps subtype.

Objectives of the present disclosure include, for example, seeking for a method for predicting efficacy of glucocorticoid with a non-invasive and economical biomarker, which can effectively save expenditure of patients and medical hygiene, guide physicians to reasonably use drugs, and further establish an accurate medical system. Through the previous work based on the proteomics technologies, the inventors found that a cysteine protease inhibitor family member Cystatin SN (CST1) can predict the sensitivity of a patient with chronic rhinosinusitis with nasal polyps to glucocorticoid. The Cystatin SN is encoded by the CST1 gene in human beings.

The present disclosure relates to use of a Cystatin SN detection agent in preparation of a kit for predicting sensitivity of a patient with chronic rhinosinusitis with nasal polyps to glucocorticoid.

The present disclosure relates to use of a Cystatin SN detection agent for predicting sensitivity of a patient with chronic rhinosinusitis with nasal polyps to glucocorticoid.

The present disclosure relates to a method for predicting sensitivity of a patient with chronic rhinosinusitis with nasal polyps to glucocorticoid, including detecting an amount of Cystatin SN in a patient sample using a Cystatin SN detection agent.

The present disclosure provides use of Cystatin SN or a coding gene CST1 thereof as a biomarker in detecting chronic rhinosinusitis with nasal polyps subtype or predicting sensitivity of a patient with chronic rhinosinusitis with nasal polyps to glucocorticoid.

The present disclosure provides use of a CST1 gene as a biomarker in detecting chronic rhinosinusitis with nasal polyps subtype.

The present disclosure provides a method for detecting chronic rhinosinusitis with nasal polyps subtype or predicting sensitivity of a patient with chronic rhinosinusitis with nasal polyps to glucocorticoid, including detecting an amount of Cystatin SN or a coding gene CST1 thereof in a patient sample.

The present disclosure provides use of CST1 or a coding gene CST1 thereof as a biomarker for detecting chronic rhinosinusitis with nasal polyps subtype or predicting sensitivity of a patient with chronic rhinosinusitis with nasal polyps to glucocorticoid.

### BRIEF DESCRIPTION OF DRAWINGS

FIG. 1 is a graph of real-time quantitative PCR amplification of a part of CST1 genes according to Experimental Example 1 of the present disclosure;

FIG. 2 is a graph of real-time quantitative PCR amplification of another part of CST1 genes according to Experimental Example 1 of the present disclosure;

FIG. 3 is a graph of real-time quantitative PCR amplification of a further part of CST1 genes according to Experimental Example 1 of the present disclosure;

FIG. 4 is a graph of real-time quantitative PCR amplification of a further part of CST1 genes according to Experimental Example 1 of the present disclosure;

FIG. 5 is a graph of melting curve of real-time quantitative PCR of a part of CST1 genes according to Experimental Example 1 of the present disclosure;

FIG. 6 is a graph of melting curve of real-time quantitative PCR of another part of CST1 genes according to Experimental Example 1 of the present disclosure;

FIG. 7 is a graph of melting curve of real-time quantitative PCR of a further part of CST1 genes according to Experimental Example 1 of the present disclosure;

FIG. 8 is a graph of melting curve of real-time quantitative PCR of a further part of CST1 genes according to Experimental Example 1 of the present disclosure;

FIG. 9 shows an alternative ROC curve of chronic rhinosinusitis with nasal polyps typing detection according to Experimental Example 1 of the present disclosure;

FIG. 10 is a graph of real-time quantitative PCR amplification of a part of CST1 genes according to Experimental Example 2 of the present disclosure;

FIG. 11 is a graph of real-time quantitative PCR amplification of another part of CST1 genes according to Experimental Example 2 of the present disclosure;

FIG. 12 is a graph of melting curve of real-time quantitative PCR of a part of CST1 genes according to Experimental Example 2 of the present disclosure;

FIG. 13 is a graph of melting curve of real-time quantitative PCR of another part of CST1 genes according to Experimental Example 2 of the present disclosure;

FIG. 14 is a graph of real-time quantitative PCR amplification of CST1 genes according to Experimental Example 3 of the present disclosure;

FIG. 15 is a graph of melting curve of real-time quantitative PCR of CST1 genes according to Experimental Example 3 of the present disclosure;

FIG. 16 is a graph of real-time quantitative PCR amplification of CST1 genes according to Experimental Example 4 of the present disclosure;

FIG. 17 is a graph of melting curve of real-time quantitative PCR of CST1 genes according to Experimental Example 4 of the present disclosure;

FIG. 18 is an ROC curve of CST1 concentration in nasal secretions and percentage of eosinophils in polyp tissues in one example of the present disclosure;

FIG. 19 is a scatter diagram of CST1 concentration in nasal secretions detected between a glucocorticoid sensitive group and a glucocorticoid insensitive group in one example of the present disclosure;

FIG. 20 is a scatter diagram of percentages of eosinophils in polyp tissues detected between a glucocorticoid sensitive group and a glucocorticoid insensitive group in one example of the present disclosure;

FIG. 21 is a graph of changes of CST1 concentration before and after a glucocorticoid sensitive group is administered with hormone in one example of the present disclosure;

FIG. 22 is a graph of changes of CST1 concentration before and after a glucocorticoid insensitive group is administered with hormone in one example of the present disclosure; and

FIG. 23 is a graph of correlation analysis of CST1 concentration in nasal secretions and percentage of eosinophils in polyp tissues in one embodiment of the present disclosure.

### DETAILED DESCRIPTION OF EMBODIMENTS

The technical solutions in the examples of the present disclosure will be described below clearly and completely, and apparently, only some but not all examples of the present disclosure are described. Based on the examples in the present disclosure, all of other examples obtained by a person ordinarily skilled in the art, without using creative effect, fall within the scope of protection of the present disclosure.

The present disclosure provides a kit for detecting chronic rhinosinusitis with nasal polyps subtype, wherein the kit includes a specific primer of a CST1 gene.

In the present disclosure, a kit for detecting chronic rhinosinusitis with nasal polyps subtype is prepared by adopting the CST1 gene as a biomarker through extensive creative experimental screening by proteomics and transcriptomics methods, and no corresponding report has been provided currently in the prior art. The CST1 gene therein is a known gene, of which the gene ID is 1469, the DNA sequence is as represented by SEQ ID NO. 1, and the gene NM No. is 001898.2.

In one or more embodiments, an upstream primer of the CST1 gene is represented by SEQ ID NO. 2, and a downstream primer of the CST1 gene is represented by SEQ ID NO. 3. With the upstream primer and the downstream primer determined in the kit of the present disclosure, the kit of the present disclosure has the highest accuracy and is more effective when identifying the nasal polyp subtype, so that the kit is suitable for mass and rapid detection.

In one or more embodiments, the kit further includes a reference gene. More preferably, the reference gene is GAPDH, an upstream primer of the reference gene is represented by SEQ ID NO. 4, and a downstream primer of the reference gene is represented by SEQ ID NO. 5. With the upstream primer and the downstream primer of the reference gene determined by the kit of the present disclosure, the kit of the present disclosure, when identifying the nasal polyp subtype, can effectively acquire a suitable ACT value by displaying the expression of the CST1 gene compared with the GAPDH, so as to identify the nasal polyp subtype, with higher accuracy.

In one or more embodiments, the kit further includes a specific primer of the reference gene.

In one or more embodiments, the reference gene is GAPDH, the upstream primer of the reference gene is represented by SEQ ID NO. 4, and the downstream primer of the reference gene is represented by SEQ ID NO. 5.

In one or more embodiments, the kit further includes: a reagent for extracting RNA from nasal polyp tissues or from nasal mucosa exfoliated cells; a reagent for making total RNA to undergo reverse transcription to cDNA; and a reagent for performing real-time quantitative PCR reaction on the CST1 gene and the reference gene in the cDNA by adopting quantitative polymerase chain reaction. More preferably, the reagent for making total RNA to undergo reverse transcription to cDNA includes: a reverse transcription mixed solution and RNase-free and DNase-free water (RNA enzyme-free and DNA enzyme-free water); the reagent for performing real-time quantitative PCR reaction on the CST1 gene and the reference gene in the cDNA by adopting quantitative polymerase chain reaction includes: a PCR premix (premixed solution), double distilled water, a machine fluorescence compensation and correction agent, an upstream primer of the CST1 gene, a downstream primer of the CST1 gene, an upstream primer of the reference gene and a downstream primer of the reference gene.

In one or more embodiments, the reagent for extracting RNA from nasal polyp tissues may be selected from following two reagents. A first reagent for extracting RNA from nasal polyp tissues includes: an RNA extraction solution, chloroform, isopropanol, ethanol with a concentration of 65%˜90%, and RNase-free and DNase-free water.

In one or more embodiments, a second reagent for extracting RNA from nasal polyp tissues includes: a cell lysis solution, a first buffer solution for removing impurities of a purification column adsorbed with RNA, a second buffer solution for removing impurities and salt of the purification column adsorbed with RNA, and RNase-free and DNase-free water; a tool for extracting RNA from nasal polyp tissues includes an RNA purification column, wherein the reagent for extracting RNA from the nasal polyp tissues further includes a DNase reaction solution (DNA enzyme reaction solution) or the tool for extracting RNA from nasal polyp tissues further includes a genomic DNA adsorption column; the DNase reaction solution includes a DNase buffer solution, recombinant DNase (recombinant DNA enzyme) and RNase-free double distilled water (RNA enzyme-free double distilled water). A person skilled in the art might make a selection according to actual preparation requirements.

In one or more embodiments, the reagent for making total RNA to undergo reverse transcription to cDNA includes: a reverse transcription mixed solution and RNase-free and DNase-free water.

In one or more embodiments, the reagent for making total RNA to undergo reverse transcription to cDNA includes: 1 μ40 μL of a reverse transcription mixed solution and 0 μL˜160 μL of RNase-free and DNase-free water. Further preferably, the reagent for making total RNA to undergo reverse transcription to cDNA includes: 2 μof the reverse transcription mixed solution and 0 μL˜8 μL of the RNase-free and DNase-free water.

In one or more embodiments, the reagent for performing real-time quantitative PCR reaction on the CST1 gene and the reference gene in the cDNA by adopting quantitative polymerase chain reaction includes: a PCR premix, double distilled water, a machine fluorescence compensation and correction agent, an upstream primer of the CST1 gene, a downstream primer of the CST1 gene, an upstream primer of the reference gene and a downstream primer of the reference gene.

In one or more embodiments, the reagent for performing real-time quantitative PCR reaction on the CST1 gene and the reference gene in the cDNA by adopting quantitative polymerase chain reaction includes: 1 μ25 μL of a PCR premix, 0 μ50 μL of double distilled water, 0 μL-2 μL of machine fluorescence compensation and correction agent, 0.01˜100 μM upstream primer of the CST1 gene, 0.01˜100 μM downstream primer of the CST1 gene, 0.01˜100 μM upstream primer of the reference gene, and 0.01˜100 μM downstream primer of the reference gene; further preferably, the reagent for performing real-time quantitative PCR reaction on the CST1 gene and the reference gene in the cDNA by adopting quantitative polymerase chain reaction includes: 5 μof a PCR premix, 0 μL˜10 μL of double distilled water, replenishing the system to 10 μL according to the total volume, 0.2 pL of machine fluorescence compensation and correction agent, 1 μM upstream primer of the CST1 gene, 1 μM downstream primer of the CST1 gene, 1 μM upstream primer of the reference gene and 1 μM downstream primer of the reference gene.

In one or more embodiments, the reagent for extracting RNA from nasal polyp tissues may be selected from following two reagents. A first reagent for extracting RNA from nasal polyp tissues includes: an RNA extraction solution, chloroform, isopropanol, ethanol with a concentration of 65%˜90%, and RNase-free and DNase-free water, wherein the first reagent for extracting RNA from nasal polyp tissues preferably includes 0.1 mL˜20 mL of an RNA extraction solution Trizol or RNAiso Blood or RNAiso Plus or other substances containing phenol, guanidinium isothiocyanate, 8-hydroxyquinoline, guanidinium isothiocyanate or β-mercaptoethanol, chloroform in a volume 0.1˜0.5 times that of the Trizol or the RNAiso Blood or the RNAiso Plus or other substances containing phenol, guanidinium isothiocyanate, 8-hydroxyquinoline, guanidinium isothiocyanate or β-mercaptoethanol, isopropanol in a volume 0.5˜3 times that of the chloroform, 65% to 90% ethanol in a volume 0.5˜5 times that of the isopropanol, and 0.01 mL to 5 mL of RNase-free and DNase-free water; further preferably, the reagent for extracting RNA from nasal polyp tissues may be selected from following two reagents, wherein a first reagent for extracting RNA from nasal polyp tissues includes: 1 mL of an RNA extraction solution Trizol or RNAiso Blood or RNAiso Plus or other substances containing phenol, guanidinium isothiocyanate, 8-hydroxyquinoline, guanidinium isothiocyanate or β-mercaptoethanol, 200 μL of chloroform, 200 μL of isopropanol, 200 μL of ethanol with a volume concentration of 65%˜90%, and 0.02 mL of RNase-free and DNase-free water; and a second reagent for extracting RNA from nasal polyp tissues includes: a cell lysis solution, a first buffer solution for removing impurities of a purification column adsorbed with RNA, a second buffer solution for removing impurities and salt of the purification column adsorbed with RNA and RNase-free and DNase-free water; a tool for extracting RNA from nasal polyp tissues includes an RNA purification column, wherein the reagent for extracting RNA from the nasal polyp tissues further includes a DNase reaction solution or the tool for extracting RNA from nasal polyp tissues further includes a genomic DNA adsorption column; and the DNase reaction solution includes a DNase buffer solution, recombinant DNase and RNase-free double distilled water.

More preferably, the second reagent for extracting RNA from nasal polyp tissues includes: 0.1 mL˜2 mL of a cell lysis solution for lysing cells and inhibiting RNA degradation, 0.1 mL˜0.7 mL of a first buffer solution for washing, 0.1 mL˜0.7 mL of a second buffer solution for washing, 0.01 mL˜1 mL of RNase-free and DNase-free water, 0˜10 μL of recombinant DNase for removing genomic DNA, 0˜10 μL of a DNase buffer solution for removing genomic DNA, and 20˜100 μL of RNase-free double distilled water, and the tool for extracting RNA from the nasal polyp tissues includes an RNA purification column; or the second reagent for extracting RNA from nasal polyp tissues includes: 0.1 mL˜2 mL of a cell lysis solution for lysing cells and inhibiting RNA degradation, 0.1 mL˜0.7 mL of a first buffer solution for washing, 0.1 mL˜0.7 mL of a second buffer solution for washing, and 0.01 mL˜1 mL of RNase-free and DNase-free water, and the tool for extracting RNA from the nasal polyp tissues includes a genomic DNA adsorption column and an RNA purification column.

Specifically, the second reagent for extracting RNA from nasal polyp tissues includes 300 μL of a cell lysis solution for lysing cells and inhibiting RNA degradation, 500 μL of a first buffer solution for washing, 600 μL of a second buffer solution for washing, 0.02 mL of RNase-free and DNase-free water, 4 μof recombinant DNase for removing genomic DNA, 5 μof 10× DNase buffer solution for removing genomic DNA, and 41 μL of RNase-free double distilled water, and the tool for extracting RNA from the nasal polyp tissues includes an RNA purification column; or the second reagent for extracting RNA from nasal polyp tissues includes: 300 μL of a cell lysis solution for lysing cells and inhibiting RNA degradation, 500 μL of a first buffer solution for washing, 600 μL of a second buffer solution for washing, and 0.02 mL of RNase-free and DNase-free water, and the tool for extracting RNA from the nasal polyp tissues includes a genomic DNA adsorption column and an RNA purification column.

In one or more embodiments, the nasal polyp tissues are nasal polyp tissues obtained by pathological biopsy of a nasal cavity, or the nasal mucosa exfoliated cells are nasal polyp cells obtained by brushing or sticking the surface of the nasal polyps.

In one or more embodiments, a data result of an amplification product is analyzed using a ΔCt(Ct(CST1)-Ct(GAPDH)) analysis method, and a threshold value compared with the ΔCt is 2.993.

In one or more embodiments, the reagent for making total RNA to undergo reverse transcription to cDNA includes: 1 μL˜40 μL of a reverse transcription mixed solution and 0 μ160 μL of RNase-free and DNase-free water. Further preferably, the reagent for making total RNA to undergo reverse transcription to cDNA includes: 2 μof a reverse transcription mixed solution and 0 μL˜8 μL of RNase-free and DNase-free water (replenishing the system to 8 μwith water according to the RNA amount). With all of the numerical ranges defined above, the step of reverse transcription can be realized, and a person skilled in the art could make a selection according to actual requirements.

In one or more embodiments, the reagent for performing real-time quantitative PCR reaction on the CST1 gene and the reference gene in the cDNA by adopting quantitative polymerase chain reaction includes: 1 μ25 μL of a PCR premix, 0 μ50 μL of double distilled water, 0 μL-2 μL of machine fluorescence compensation and correction agent, 0.01˜100 μM upstream primer of the CST1 gene, 0.01˜100 μM downstream primer of the CST1 gene, 0.01˜100 μM upstream primer of the reference gene and 0.01100 04 downstream primer of the reference gene; further preferably, the reagent for performing real-time quantitative PCR reaction on the CST1 gene and the reference gene in the cDNA by adopting quantitative polymerase chain reaction includes: 5 μof a PCR premix, 0 μ10 μL of double distilled water (replenishing the system to 10 μL with water according to the total volume), 0.2 μL of machine fluorescence compensation and correction agent, 1 μM upstream primer of the CST1 gene, 1 μM downstream primer of the CST1 gene, 1 μM upstream primer of the reference gene and 1 μM downstream primer of the reference gene. With all of the numerical ranges defined above, the step of performing real-time quantitative PCR amplification on the CST1 gene and the reference gene in the cDNA through the quantitative polymerase chain reaction can be realized, and a person skilled in the art could make a selection according to actual requirements.

In one or more embodiments, the reagent for extracting RNA from nasal polyp tissues may be selected from following two reagents. A first reagent for extracting RNA from nasal polyp tissues includes 0.1 mL˜20 mL of an RNA extraction solution Trizol or RNAiso Blood or RNAiso Plus or other substances containing phenol, guanidinium isothiocyanate, 8-hydroxyquinoline, guanidinium isothiocyanate or 0-mercaptoethanol, chloroform in a volume 0.1˜0.5 times that of the Trizol or the RNAiso Blood or the RNAiso Plus or the other substances containing phenol, guanidinium isothiocyanate, 8-hydroxyquinoline, guanidinium isothiocyanate or 0-mercaptoethanol, isopropanol in a volume 0.5˜3 times that of the chloroform, 65% to 90% ethanol in a volume 0.5˜5 times that of the isopropanol, and 0.01 mL to 5 mL of RNase-free and DNase-free water.

A second reagent for extracting RNA from nasal polyp tissues includes: 0.1 mL˜2 mL of a cell lysis solution for lysing cells and inhibiting RNA degradation, 0.1 mL˜0.7 mL of a first buffer solution for washing, 0.1 mL˜0.7 mL of a second buffer solution for washing, 0.01 mL˜1 mL of RNase-free and DNase-free water, 0˜10 μL of recombinant DNase for removing genomic DNA, 0˜10 μL of a DNase buffer solution for removing genomic DNA, and 20100 μL of RNase-free double distilled water, and the tool for extracting RNA from the nasal polyp tissues includes an RNA purification column; or the second reagent for extracting RNA from nasal polyp tissues includes: 0.1 mL˜2 mL of a cell lysis solution for lysing cells and inhibiting RNA degradation, 0.1 mL˜0.7 mL of a first buffer solution for washing, 0.1 mL˜0.7 mL of a second buffer solution for washing, and 0.01 mL˜1 mL of RNase-free and DNase-free water, and the tool for extracting RNA from the nasal polyp tissues includes a genomic DNA adsorption column and an RNA purification column.

In one or more embodiments, the reagent for extracting RNA from nasal polyp tissues may be selected from following two reagents. A first reagent for extracting RNA from nasal polyp tissues includes: 1 mL of an RNA extraction solution Trizol or RNAiso Blood or RNAiso Plus or other substances containing phenol, guanidinium isothiocyanate, 8-hydroxyquinoline, guanidinium isothiocyanate or 0-mercaptoethanol, 200 μL of chloroform, 200 μL of isopropanol, 200 μL of ethanol with a volume concentration of 65% to 90%, and 0.02 mL of RNase-free and DNase-free water.

A second reagent for extracting RNA from nasal polyp tissues includes: 300 μL of a cell lysis solution for lysing cells and inhibiting RNA degradation, 500 μL of a first buffer solution for washing, 600 μL of a second buffer solution for washing, 0.02 mL of RNase-free and DNase-free water, 4 μof recombinant DNase for removing genomic DNA, 5 μ10x DNase buffer solution for removing genomic DNA, and 41 μL of RNase-free double distilled water, and the tool for extracting RNA from the nasal polyp tissues includes an RNA purification column; or the second reagent for extracting RNA from nasal polyp tissues includes: 300 μL of a cell lysis solution for lysing cells and inhibiting RNA degradation, 500 μL of a first buffer solution for washing, 600 μL of a second buffer solution for washing, and 0.02 mL of RNase-free and DNase-free water, and the tool for extracting RNA from the nasal polyp tissues includes a genomic DNA adsorption column and an RNA purification column. With all of the numerical ranges defined above, the step of extracting RNA from nasal polyp tissues can be realized, and a person skilled in the art could make a selection according to actual requirements. In the above, the RNA extraction solution is Trizol, RNAiso Blood and RNAiso Plus, all of which are trade names.

In the above, the cell lysis solution is used to rapidly break off cells and inhibit substances of nuclease released by the cells, the first buffer solution is used to remove impurities of a purification column adsorbed with RNA, the second buffer is used to remove impurities and salt of the purification column adsorbed with RNA, and the RNase-free and DNase-free water is used to dissolve RNA.

In one or more embodiments, the reagent for extracting RNA from nasal polyp tissues, the reagent for making total RNA to undergo reverse transcription to cDNA, and the reagent for performing real-time quantitative PCR reaction on the CST1 gene and the reference gene in the cDNA by adopting quantitative polymerase chain reaction in the above are separately packaged, respectively.

In one or more embodiments, the nasal polyp tissues are nasal polyp tissues obtained by pathological biopsy of a nasal cavity, or the nasal mucosa exfoliated cells are nasal polyp cells obtained by brushing or sticking the surface of the nasal polyps. In the above, the brushing or sticking manner avoids causing wounds to the patient, improves the safety of patient examination, and is more convenient to operate, thus saving labor costs and medical costs.

In one or more embodiments, a data result of an amplification product is analyzed using a ΔCt (Ct(CST1)-Ct(GAPDH)) analysis method, and a threshold value compared with the ΔCt is 2.993. Defining this threshold value can enable the accuracy of the kit provided in the present disclosure in detecting chronic rhinosinusitis with nasal polyps subtype to reach no less than 85%.

An example of the present disclosure further provides use of a CST1 gene as a biomarker in preparation of a product for detecting chronic rhinosinusitis with nasal polyps subtype. In the above, the product may be a detection reagent, a chip or a kit. Although only the specific technical contents of the kit are illustrated in the above examples, a person skilled in the art could directly obtain the specific technical contents of the detection reagent and the chip products on the basis of disclosing the technical solutions of the present disclosure in combination with general common knowledge.

The present disclosure provides use of a CST1 gene as a biomarker in preparation of a product for detecting chronic rhinosinusitis with nasal polyps subtype.

The present disclosure provides a method for detecting an expression level of a CST1 gene in nasal brushing cells, including steps of: extracting RNA from nasal brushing cells, making total RNA to undergo reverse transcription to cDNA, performing real-time quantitative PCR amplification on a CST1 gene and a reference gene in the cDNA by using a specific primer of the CST1 gene and a specific primer of the reference gene, respectively, by adopting quantitative polymerase chain reaction, and calculating the expression level of the CST1 gene based on a detection result of an amplification product.

In one or more embodiments, the upstream primer of the CST1 gene is represented by SEQ ID NO. 2, and the downstream primer of the CST1 gene is represented by SEQ ID NO. 3.

In one or more embodiments, the reference gene is GAPDH, the upstream primer of the reference gene is represented by SEQ ID NO. 4, and the downstream primer of the reference gene is represented by SEQ ID NO. 5.

In one or more embodiments, the nasal brushing cells are obtained from the surface of nasal polyps with a brush, and the brush is stored in a cell lysis solution at a temperature below 4° C. after the nasal brushing cells are obtained.

In one or more embodiments, there are two methods for extracting RNA from the nasal brushing cells, wherein a first method includes the following steps:

Step 1: dissolving the nasal brushing cells in 1002000 uL of a cell lysis solution, adding an equal volume of ethanol, mixing them uniformly and then adding the resultant to an RNA purification column, removing a filtrate in a collection tube after centrifugal treatment, and placing the RNA purification column into the collection tube; and

Step 2: adding 300 uL-700 uL of a first buffer solution to the RNA purification column obtained in step 1, and removing a first filtrate after centrifugal treatment; continuously adding 400 uL-800 uL of a second buffer solution to the RNA purification column, removing a second filtrate after centrifugal treatment, and eluting the RNA purification column to obtain RNA.

In one or more embodiments, the method for extracting RNA from nasal brushing cells further includes the following steps: adding 10100 ut of a DNase reaction solution to the RNA purification column from which the second filtrate is removed, standing, then adding 300 uL˜700 uL of the second buffer solution, removing a third filtrate after centrifugal treatment, eluting the RNA purification column, and then measuring RNA purity using a spectrophotometer to obtain RNA.

A method for preparing the DNase reaction solution includes the following step: mixing a DNase buffer solution, recombinant DNase and RNase-free double distilled water to obtain the DNase reaction solution. Preferably, the method for preparing the DNase reaction solution includes the following step: mixing 5 μof 10× DNase buffer solution, 4 μof the recombinant DNase, and 41 uL of RNase-free double distilled water to obtain the DNase reaction solution.

In one or more embodiments, when RNA extraction is performed with a low genome content or a small starting amount of material, the method for extracting RNA from nasal brushing cells further includes the following step: in the step 1, dissolving the nasal brushing cells in a cell lysis solution and then adding the resultant to a genomic DNA adsorption column to obtain a filtrate, and then adding an equal volume of ethanol to the filtrate.

In one or more embodiments, in order to obtain a higher concentration of RNA, the method for extracting RNA from nasal brushing cells further includes the following steps: adding RNase-free (RNA hydrolase-free) distilled water or diethyl pyrocarbonate treated water to the RNA purification column to be eluted, after standing at room temperature, centrifuging the resultant and eluting the RNA purification column, and measuring RNA purity using a spectrophotometer to obtain RNA.

In one or more embodiments, in step 1, the cell lysis solution can rapidly break off the nasal brushing cells and inhibit substances of nuclease released by the nasal brushing cells; the genomic DNA is removed using the genomic DNA adsorption column; in step 2, the RNA purification column is used to enrich RNA, wherein the collection tube is used for collecting a solution after removal of genomic DNA, the first buffer solution for removing impurities of the purification column adsorbed with RNA, and the second buffer solution for removing impurities and salt in the RNA solution.

In one or more embodiments, a first method for extracting RNA from nasal brushing cells includes the following steps:

Step 1: dissolving the nasal brushing cells in 300 μL of a cell lysis solution, adding an equal volume of 70% ethanol, and mixing the solution uniformly using a pipette gun; immediately adding the mixed solution to an RNA purification column, centrifuging the resultant at 12000 r/min for 1 min, removing a filtrate, and placing the RNA purification column in a 2 mL collection tube;

Step 2: adding 500 μL of a first buffer solution to the RNA purification column obtained in step 1, centrifuging the resultant at 12000 r/min for 30 s, and removing a first filtrate; adding 600 μL of a second buffer solution to the RNA purification column continuously, centrifuging the resultant at 12000 r/min for 30 s, and removing a second filtrate;

Step 3: mixing 5 μof 10x DNase buffer solution, 4 μof the recombinant DNase, and 41 μL of the RNase-free double distilled water to obtain the DNase reaction solution, adding 50 μL of the DNase reaction solution to the RNA purification column from which the second filtrate is removed, standing at room temperature for 15 minutes, adding 350 μL of the second buffer solution, centrifuging the resultant at 12000 r/min for 30 seconds, and removing a third filtrate; and

Step 4: positioning the RNA purification column from which the third filtrate is removed in step 3 in a 1.5 mL RNase-free collection tube, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA when a ratio of OD260/OD280 of the RNA solution is measured to be 1.7˜2.1 using a spectrophotometer.

In one or more embodiments, the method for extracting RNA from nasal brushing cells includes the following steps:

Step 1: placing the genomic DNA adsorption column in a 2 mL collection tube, dissolving the nasal brushing cells in 1002000 μL of the cell lysis solution and then adding the resultant to the genomic DNA adsorption column, taking a filtrate, adding an equal volume of 70% ethanol to the filtrate, after mixing them uniformly, adding the resultant to an RNA purification column, centrifuging the resultant at 12000 r/min for 1 min, removing a filtrate, and placing the RNA purification column in a 2 mL collection tube;

Step 2: adding 500 μL of a first buffer solution to the RNA purification column obtained in step 1, centrifuging the resultant at 12000 r/min for 30 s, and removing a first filtrate; adding 600 μL of a second buffer solution to the RNA purification column continuously, centrifuging the resultant at 12000 r/min for 30 s, and removing a second filtrate; and

Step 3: positioning the RNA purification column from which the second filtrate is removed in step 2 in a 1.5 mL RNase-free collection tube, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA when a ratio of OD260/OD280 of the RNA solution is measured to be 1.7˜2.1 using a spectrophotometer.

In one or more embodiments, a second method for extracting RNA from nasal brushing cells includes the following steps: adding 0.1 mL˜20 mL of an RNA extraction solution to a centrifuge tube with the nasal brushing cells to dissolve and shake the nasal brushing cells, then adding chloroform in a volume 0.10˜0.5 times that of the RNA extraction solution, shaking the centrifuge tube to mix them uniformly, standing at room temperature, centrifuging the resultant, taking a supernatant, adding isopropanol in a volume 0.5˜3 times that of the chloroform, mixing them uniformly, then standing and centrifuging, discarding the supernatant, retaining a first precipitate, adding ethanol with a concentration of 65° 4˜90% in a volume 0.5˜5 times that of the isopropanol to the first precipitate, after washing, mixing them uniformly and centrifuging, discarding the supernatant, and retaining a second precipitate; capping the centrifuge tube tightly, centrifuging again, removing the supernatant, and then continuously adding 0.015 mL of RNase-free and DNase-free water to the centrifuge tube to dissolve the second precipitate, and measuring RNA purity using a spectrophotometer to obtain RNA.

In one or more embodiments, the RNA extraction solution is Trizol, RNAiso Blood, RNAiso Plus or other reagents containing any one or more of phenol, guanidinium isothiocyanate, 8-hydroxyquinoline, guanidinium isothiocyanate and 0-mercaptoethanol.

In one or more embodiments, the second method for extracting RNA from nasal brushing cells includes the following steps: adding 0.120 mL of an RNA extraction solution to a centrifuge tube with the nasal brushing cells to dissolve and shake the nasal brushing cells, and then standing at room temperature for 3˜7 min; adding 40 μL-5 mL of chloroform, shaking the centrifuge tube to mix them uniformly, standing at room temperature for 3˜7 min, centrifuging the resultant at 1000014000 r/min at 3° C.˜5° C. for 1020 min; taking 40 μL-8 mL of a supernatant, adding an equal volume of isopropanol, mixing them uniformly, then standing for 8˜12 min, centrifuging the resultant at 1000014000 r/min at 3° C.˜5° C. for 1020 min, discarding the supernatant, and retaining a first precipitate; adding ethanol with a concentration of 65° 4˜90% in a volume equal to that of the isopropanol to the first precipitate, centrifuging the resultant at 700014000 r/min at 3° C.˜5° C. for 1020 min, discarding the supernatant, and retaining a second precipitate; capping the centrifuge tube tightly, centrifuging the resultant at 700014000 r/min at 3° C.˜5° C. for 1˜3 min, removing the supernatant, standing for 1020 min, and then continuously adding 0.015 mL of RNase-free and DNase-free water to the centrifuge tube to dissolve the second precipitate, and measuring RNA purity using a spectrophotometer to obtain RNA.

In one or more embodiments, the second method for extracting RNA from nasal brushing cells includes the following steps: adding 1 mL of an RNA extraction solution to a centrifuge tube with the nasal brushing cells to dissolve and shake the nasal brushing cells, and then standing at room temperature for 5 min; adding 200 μL of chloroform, shaking the centrifuge tube to mix them uniformly, standing at room temperature for 5 min, centrifuging the resultant at 12000 r/min at 4° C. for 15 min; taking 200 μL of a supernatant, adding 200 pL of isopropanol, mixing them uniformly, then standing for 10 min, centrifuging the resultant at 12000 r/min at 4° C. for 15 min, discarding the supernatant, and retaining a first precipitate; adding ethanol with a concentration of 75% in a volume equal to that of the isopropanol to the first precipitate, centrifuging the resultant at 7500 r/min at 4° C. for 15 min, discarding the supernatant, and retaining a second precipitate; capping the centrifuge tube tightly, centrifuging the resultant at 7500 r/min at 4° C. for 2 min, removing the supernatant, standing for 15 min, and then continuously adding 50 μL of RNase-free and DNase-free water to the centrifuge tube to dissolve the second precipitate, and obtaining RNA when a ratio of OD260/OD280 of the RNA solution is measured to be 1.7˜2.1 using a spectrophotometer.

In one or more embodiments, a method for reverse transcription of total RNA to cDNA includes following steps: making 1˜3 μL of a reverse transcription mixed solution, 0˜10 μL of RNase-free distilled water and the extracted RNA to undergo reverse transcription under a temperature condition of 37° C. for 15 min, and then carrying out inactivation reaction of reverse transcriptase under a temperature condition of 84° C., to obtain a reverse transcription product cDNA.

In one or more embodiments, the method for reverse transcription of total RNA to cDNA includes following steps: taking 2 μof the reverse transcription mixed solution, 8 μof the RNase-free distilled water, and the total RNA in a total amount not exceeding 500 ng or in a volume not exceeding 8 μ, replenishing the system to 10 μL with the RNase-free distilled water; gently mixing them uniformly, and then conducting reverse transcription, under following condition: conducting the reverse transcription under a condition of 37° C. for 15 min; conducting the inactivation reaction of reverse transcriptase under a condition of 85° C. for 5 seconds; and leaving the product at 4° C. In the above, the reaction system may be correspondingly amplified according to requirements, wherein a maximum of 500 ng of total RNA may be used in a 10 μL reaction system, and a person skilled in the art might make a selection according to actual preparation requirements.

In one or more embodiments, the real-time quantitative PCR amplification includes the following steps:

Step 1: preparing a real-time quantitative PCR reaction solution: including 1 pL-25 μL of a PCR premix, 0 μ10 μL of double distilled water, replenishing the system to 10 μL according to the total volume, 0 μL-2 μL of machine fluorescence compensation and correction agent, 0.01˜100 μM upstream primer of the CST1 gene, 0.01˜100 μM downstream primer of the CST1 gene, 0.01˜100 μM upstream primer of the reference gene, 0.01˜100 μM downstream primer of the reference gene, and 0.01 μL-5 μL of cDNA;

Step 2: performing real-time quantitative PCR detection by adopting a two-step PCR amplification standard procedure or a three-step PCR amplification standard procedure; and

Step 3: calculating the expression level of the CST1 gene.

In one or more embodiments, preparing the real-time quantitative PCR reaction solution includes: 5 μof the PCR premix, 2.8 μL of double distilled water, replenishing the system to 10 μL according to the total volume, 0.2 μL of machine fluorescence compensation and correction agent, 0.5 μL of an upstream primer of the CST1 gene, 0.5 μL of a downstream primer of the CST1 gene, 0.5 μL of an upstream primer of the reference gene, 0.5 μL of a downstream primer of the reference gene, and 1 ng/μL of the cDNA or 0.01 μL to 5 μL of the RNA.

In one or more embodiments, reaction condition of the two-step PCR amplification standard procedure includes the following steps: in stage 1: pre-denaturation under a condition of 95° C. for 30 seconds; in stage 2: PCR reaction under a condition of 95° C. for 15 seconds, and under a condition of 60° C. for 60 seconds, annealing and extension, 40 cycles in total as such; and

Reaction condition of the three-step PCR amplification standard procedure include the following steps: in stage 1: pre-denaturation under a condition of 95° C. for 2 minutes; in stage 2: PCR reaction under a condition of 95° C. for 1 minute, under a condition of 55° C. for 1 minute, and under a condition of 72° C. for 1 minute, which are carried out for 40 cycles as such; and finally, annealing and extension at 72° C. for 7 minutes.

A method for calculating the expression level of the CST1 gene is: calculating ΔCT or 2^{-AACT} to calculate the expression level of target gene, wherein ΔCT=(CT(CST1)-CT(GAPDH)), −ΔΔCT=-(ACT(treated specimen CT(CST1)-CT(GAPDH))-healthy control group mean ACT); healthy control group mean ΔCT=/each control group ΔCT(CT(CST1)-CT(GAPDH))/number of samples in control group.

An example of the present disclosure provides a method for detecting expression level of a CST1 gene in nasal brushing cells, including steps of: extracting RNA from nasal brushing cells, making total RNA to undergo reverse transcription to cDNA, performing real-time quantitative PCR amplification on a CST1 gene and a reference gene in the cDNA by using a specific primer of the CST1 gene and a specific primer of the reference gene, respectively, by adopting quantitative polymerase chain reaction, and calculating the expression level of the CST1 gene based on a detection result of an amplification product.

In the present disclosure, the chronic rhinosinusitis with nasal polyps subtype is detected by calculating the expression level of the CST1 gene through extensive creative experimental screening by proteomics and transcriptomics methods, and the method provided for calculating the expression level of the CST1 gene is simple and reliable, with high accuracy. No corresponding report has been provided currently in the prior art. The CST1 gene involved is a known gene, of which the gene ID is 1469, and the DNA sequence is represented by SEQ ID NO. 1.

In one or more embodiments, the upstream primer of the CST1 gene is represented by SEQ ID NO. 2, and the downstream primer of the CST1 gene is represented by SEQ ID NO. 3. For the design of the upstream primer and the downstream primer of the CST1 gene, the sensitivity is higher, and when the expression level of the CST1 gene is detected, the result is more accurate and the repeatability is better.

In one or more embodiments, the reference gene is GAPDH, the upstream primer of the reference gene is represented by SEQ ID NO. 4, and the downstream primer of the reference gene is represented by SEQ ID NO. 5. With regard to the design of this feature, in combination with the upstream primer of the CST1 gene and the downstream primer of the CST1 gene mentioned above, an appropriate ACT value is obtained, so as to calculate the expression level of the CST1 gene, and have higher accuracy.

In one or more embodiments, the nasal brushing cells are obtained from the surface of the nasal polyps with a brush, and the brush is stored in a cell lysis solution at a temperature below 4° C. after the nasal brushing cells are obtained. Based on this manner, the method of the present disclosure avoids causing wounds to the patient, improves the safety of patient examination, and is more convenient to operate, thus saving labor costs and medical costs.

In one or more embodiments, there are two methods for extracting RNA from the nasal brushing cells, wherein a first method includes the following steps:

Step 1: dissolving the nasal brushing cells in 1002˜000 μL of the cell lysis solution, adding an equal volume of ethanol, mixing them uniformly and then adding the resultant to an RNA purification column, removing a filtrate in a collection tube after centrifugal treatment, and placing the RNA purification column into the collection tube; and

Step 2: adding 300 μL-700 μL of a first buffer solution to the RNA purification column obtained in step 1, and removing a first filtrate after centrifugal treatment; continuously adding 400 μL-800 μL of the second buffer solution to the RNA purification column, removing a second filtrate after centrifugal treatment, and eluting the RNA purification column to obtain RNA.

In one or more embodiments, the method for extracting RNA from nasal brushing cells further includes the following step: adding 10100 μL of the DNase reaction solution to the RNA purification column from which the second filtrate is removed, standing, then adding 300 μL˜700 μL of the second buffer solution, removing a third filtrate after centrifugal treatment, eluting the RNA purification column, and then measuring RNA purity using a spectrophotometer to obtain RNA.

In one or more embodiments, the method for preparing the DNase reaction solution includes the following step: mixing a DNase buffer solution, recombinant DNase, and RNase-free double distilled water to obtain the DNase reaction solution. Preferably, the method for preparing the DNase reaction solution includes the following step: mixing 5 μof 10× DNase buffer solution, 4 μof the recombinant DNase, and 41 μL of RNase-free double distilled water to obtain the DNase reaction solution.

In one or more embodiments, when RNA extraction is performed with a low genome content or a small starting amount of material, the method for extracting RNA from nasal brushing cells further includes the following step: in the step 1, dissolving the nasal brushing cells in a cell lysis solution and then adding the resultant to a genomic DNA adsorption column to obtain a filtrate, and then adding an equal volume of ethanol to the filtrate.

In one or more embodiments, in order to obtain a higher concentration of RNA, the method for extracting RNA from nasal brushing cells further includes the following steps: adding RNase-free distilled water or diethyl pyrocarbonate treated water to the RNA purification column to be eluted, after standing at room temperature, centrifuging and eluting the RNA purification column, and measuring RNA purity using a spectrophotometer to obtain RNA.

In one or more embodiments, in step 1, the cell lysis solution can rapidly break off the nasal brushing cells and inhibit substances of nuclease released by the nasal brushing cells; genomic DNA adsorption column is used for removing genomic DNA; in step 2, the RNA purification column is used to enrich RNA, wherein the collection tube is used for collecting a solution after removal of genomic DNA, the first buffer solution for removing impurities of the purification column adsorbed with RNA, and the second buffer solution for removing impurities and salt in the RNA solution.

In one or more embodiments, a first method for extracting RNA from nasal brushing cells includes the following steps:

Step 1: dissolving the nasal brushing cells in 300 μL of a cell lysis solution, adding an equal volume of 70% ethanol, and mixing the solution uniformly using a pipette gun; immediately adding the mixed solution to an RNA purification column, centrifuging the resultant at 12000 r/min for 1 min, removing a filtrate, and placing the RNA purification column in a 2 mL collection tube;

Step 2: adding 500 μL of a first buffer solution to the RNA purification column obtained in step 1, centrifuging the resultant at 12000 r/min for 30 s, and removing a first filtrate; adding 600 μL of a second buffer solution to the RNA purification column continuously, centrifuging the resultant at 12000 r/min for 30 s, and removing a second filtrate;

Step 3: mixing 5 μof 10x DNase buffer solution, 4 μof the recombinant DNase and 41 μL of the RNase-free double distilled water to obtain the DNase reaction solution, adding 50 μL of the DNase reaction solution to the RNA purification column from which the second filtrate is removed, standing at room temperature for 15 minutes, adding 350 μL of the second buffer solution, centrifuging the resultant at 12000 r/min for 30 s, and removing a third filtrate; and

Step 4: positioning the RNA purification column from which the third filtrate is removed in step 3 in a 1.5 mL RNase-free collection tube, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA when a ratio of OD260/OD280 of the RNA solution is measured to be 1.7˜2.1 using a spectrophotometer.

In one or more embodiments, the method for extracting RNA from nasal brushing cells includes the following steps:

Step 1: placing the genomic DNA adsorption column in a 2 mL collection tube, dissolving the nasal brushing cells in 100˜2000 μL of the cell lysis solution and then adding the resultant to the genomic DNA adsorption column, taking a filtrate, adding an equal volume of 70% ethanol to the filtrate, after mixing them uniformly, adding the resultant to an RNA purification column, centrifuging the resultant at 12000 r/min for 1 min, removing a filtrate, and placing the RNA purification column in a 2 mL collection tube;

Step 2: adding 500 μL of a first buffer solution to the RNA purification column obtained in step 1, centrifuging the resultant at 12000 r/min for 30 s, and removing a first filtrate; adding 600 μL of a second buffer solution to the RNA purification column continuously, centrifuging the resultant at 12000 r/min for 30 s, and removing a second filtrate; and

Step 3: positioning the RNA purification column from which the second filtrate is removed in step 2 in a 1.5 mL RNase-free collection tube, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA when a ratio of OD260/OD280 of the RNA solution is measured to be 1.7˜2.1 using a spectrophotometer.

In one or more embodiments, in step 1, the cell lysis solution can rapidly break off the nasal brushing cells and inhibit substances of nuclease released by the nasal brushing cells; in step 1, the genomic DNA is removed using the genomic DNA adsorption column; in step 2, the RNA purification column is used to enrich RNA, wherein the collection tube is used for collecting a solution after removal of genomic DNA, the first buffer solution for removing impurities of the purification column adsorbed with RNA, and the second buffer solution for removing impurities and salt in the RNA solution.

In one or more embodiments, a second method for extracting RNA from nasal brushing cells includes the following steps: adding 0.1 mL˜20 mL of an RNA extraction solution to a centrifuge tube with the nasal brushing cells to dissolve and shake the nasal brushing cells, then adding chloroform in a volume 0.10˜0.5 times that of the RNA extraction solution, shaking the centrifuge tube to mix them uniformly, standing at room temperature, centrifuging the resultant, taking a supernatant, adding isopropanol in a volume 0.5˜3 times that of the chloroform, mixing them uniformly, then standing and centrifuging, discarding a supernatant, retaining a first precipitate, adding ethanol with a concentration of 65%˜90% in a volume 0.5˜5 times that of the isopropanol to the first precipitate, after washing, mixing them uniformly and centrifuging, discarding the supernatant, and retaining a second precipitate; capping the centrifuge tube tightly, centrifuging again, removing the supernatant, and then continuously adding 0.01˜5 mL of RNase-free and DNase-free water to the centrifuge tube to dissolve the second precipitate, and measuring RNA purity using a spectrophotometer to obtain RNA.

In one or more embodiments, the RNA extraction solution is Trizol, RNAiso Blood, RNAiso Plus, or other reagents containing any one or more of phenol, guanidinium isothiocyanate, 8-hydroxyquinoline, guanidinium isothiocyanate and 0-mercaptoethanol.

In one or more embodiments, the second method for extracting RNA from nasal brushing cells includes the following steps: adding 0.120 mL of an RNA extraction solution to a centrifuge tube with the nasal brushing cells to dissolve and shake the nasal brushing cells, and then standing at room temperature for 3˜7 min; adding 40 μL-5 mL of chloroform, shaking the centrifuge tube to mix them uniformly, standing at room temperature for 3˜7 min, centrifuging the resultant at 1000014000 r/min at 3° C.˜5° C. for 1020 min; taking 40 μL-8 mL of a supernatant, adding an equal volume of isopropanol, mixing them uniformly, then standing for 8˜12 min, centrifuging the resultant at 10000˜14000 r/min at 3° C.˜5° C. for 10˜20 min, discarding the supernatant, and retaining a first precipitate; adding ethanol with a concentration of 65° 4˜90% in a volume equal to that of the isopropanol to the first precipitate, centrifuging the resultant at 700014000 r/min at 3° C.˜5° C. for 1020 min, discarding the supernatant, and retaining a second precipitate; capping the centrifuge tube tightly, centrifuging the resultant at 700014000 r/min at 3° C.˜5° C. for 1˜3 min, removing the supernatant, standing for 1020 min, and then continuously adding 0.015 mL of RNase-free and DNase-free water to the centrifuge tube to dissolve the second precipitate, and measuring RNA purity using a spectrophotometer to obtain RNA.

In one or more embodiments, the second method for extracting RNA from nasal brushing cells includes the following steps: adding 1 mL of an RNA extraction solution to a centrifuge tube with the nasal brushing cells to dissolve and shake the nasal brushing cells, and then standing at room temperature for 5 min; adding 200 μL of chloroform, shaking the centrifuge tube to mix them uniformly, standing at room temperature for 5 min, centrifuging the resultant at 12000 r/min at 4° C. for 15 min; taking 200 μL of a supernatant, adding 200 pL of isopropanol, mixing them uniformly, then standing for 10 min, centrifuging the resultant at 12000 r/min at 4° C. for 15 min, discarding the supernatant, and retaining a first precipitate; adding ethanol with a concentration of 75% in a volume equal to that of the isopropanol to the first precipitate, centrifuging the resultant at 7500 r/min at 4° C. for 15 min, discarding the supernatant, and retaining a second precipitate; capping the centrifuge tube tightly, centrifuging the resultant at 7500 r/min at 4° C. for 2 min, removing the supernatant, standing for 15 min, and then continuously adding 50 μL of RNase-free and DNase-free water to the centrifuge tube to dissolve the second precipitate, and obtaining RNA when a ratio of OD260/OD280 of the RNA solution is measured to be 1.7˜2.1 using a spectrophotometer.

In one or more embodiments, a method for reverse transcription of total RNA to cDNA includes following steps: making 1˜3 μL of a reverse transcription mixed solution, 0˜10 μL of RNase-free distilled water and the extracted RNA to undergo reverse transcription under a temperature condition of 37° C. for 15 min, and then carrying out inactivation reaction of reverse transcriptase under a temperature condition of 84° C., to obtain a reverse transcription product cDNA.

In one or more embodiments, the method for reverse transcription of total RNA to cDNA includes following steps: taking 2 μof the reverse transcription mixed solution, 8 μof the RNase-free distilled water, and the total RNA in a total amount not exceeding 500 ng or in a volume not exceeding 8 μ, replenishing the system to 10 μL with the RNase-free distilled water; gently mixing them uniformly, and then conducting reverse transcription, under following condition: conducting the reverse transcription under a condition of 37° C. for 15 min; conducting the inactivation reaction of reverse transcriptase under a condition of 85° C. for 5 seconds; and leaving a product at 4° C.

In one or more embodiments, the real-time quantitative PCR amplification includes the following steps:

Step 1: preparing a real-time quantitative PCR reaction solution: including 1 μL-25 μL of a PCR premix, 0 μ10 μL of double distilled water, replenishing the system to 10 μL according to the total volume, 0 μL-2 μL of machine fluorescence compensation and correction agent, 0.01100 pIVI upstream primer of the CST1 gene, 0.01˜100 ∞M downstream primer of the CST1 gene, 0.01˜100 μM upstream primer of the reference gene, 0.01˜100 μM downstream primer of the reference gene, and 0.01 μL-5 μL of cDNA;

Step 2: performing real-time quantitative PCR detection by adopting a two-step PCR amplification standard procedure or a three-step PCR amplification standard procedure; and

Step 3: calculating the expression level of the CST1 gene.

In one or more embodiments, preparing the real-time quantitative PCR reaction solution includes: 5 μof the PCR premix, 2.8 μL of double distilled water, replenishing the system to 10 μL according to the total volume, 0.2 μL of machine fluorescence compensation and correction agent, 0.5 μL of an upstream primer of the CST1 gene, 0.5 μL of a downstream primer of the CST1 gene, 0.5 μL of an upstream primer of the reference gene, 0.5 μL of a downstream primer of the reference gene, and 1 ng/μL of the cDNA.

In one or more embodiments, reaction condition of the two-step PCR amplification standard procedure includes the following steps: in stage 1: pre-denaturation under a condition of 95° C. for 30 seconds; in stage 2: PCR reaction under a condition of 95° C. for 15 seconds, and under a condition of 60° C. for 60 seconds, annealing and extension, 40 cycles in total as such.

Reaction condition of the three-step PCR amplification standard procedure includes the following steps: in stage 1: pre-denaturation under a condition of 95° C. for 2 minutes; in stage 2: PCR reaction under a condition of 95° C. for 1 minute, under a condition of 55° C. for 1 minute, and under a condition of 72° C. for 1 minute, which are carried out for 40 cycles as such; and finally, annealing and extension at 72° C. for 7 minutes.

A method for calculating the expression level of the CST1 gene is: calculating ΔCT or 2^{-ΔΔCT} to calculate the expression level of target gene, wherein ΔCT=(CT(CST1)-CT(GAPDH)), −ΔΔCT=-(ΔCT(treated specimen CT(CST1)-CT(GAPDH))-healthy control group mean ΔCT); healthy control group mean ΔCT=/each control group ΔCT (CT(CST1)-CT(GAPDH))/number of samples in control group. Using the relative quantification method of the ΔCT method or the 2^{-ΔΔCt} method, the reference gene with a relatively constant expression level is selected, to normalize with the number of reference genes, and the expression level of the target gene is calculated by measuring the difference between the Ct values of the target gene of the sample and the reference gene. The method is simple and fast, with high detection accuracy, can reduce the detection cost, save the detection time, and has advantages such as easy interpretation of result, thus greatly improving the experimental efficiency.

Specifically, for the difference in the expression level of CST1 and the reference gene in the same subject, the reference gene is a gene whose expression is relatively stable in vivo, and usually will not change with disease or the like, therefore comparing with the reference gene can reflect the relative abundance of the target gene and the reference gene, and thus the ACT method can be adopted. The 2^{-ΔΔCt} method may be employed for differences in the expression levels of the CST1 and the reference gene in different subjects.

Use of the above method for detecting the expression level of CST1 gene in nasal brushing cells in preparation of a kit for detecting chronic rhinosinusitis with nasal polyps subtype.

The present disclosure relates to use of a Cystatin SN detection agent in preparation of a kit for predicting sensitivity of a patient with chronic rhinosinusitis with nasal polyps to glucocorticoid.

In one or more embodiments, the kit further includes a sample pretreatment reagent.

In one or more embodiments, the sample is selected from nasal secretions and/or nasal exfoliated cells.

The sample is selected from nasal secretions and/or nasal exfoliated cells, the sample is collected safely without invasiveness, and has good tolerance, and the detection method is simple and fast.

The sample pretreatment reagent may include a sample extraction reagent (e.g., a reagent for nasal meatus sampling or expansion sponge absorption), or a protein concentration detection reagent (e.g., BCA), or a protein diluent (e.g., PBS or water, etc.).

In one or more embodiments, the kit further includes a reagent for detecting the percentage of eosinophils.

In one or more embodiments, the dosage form of the glucocorticoid includes any one of oral dosage form (e.g., oral methylprednisolone or dexamethasone phosphate tablet), injection (e.g., hydrocortisone injection), ointment (e.g., 0.1% or other concentration of betamethasone valerate ointment), spray (e.g., glucocorticoid nasal spray) and an inhalant (e.g., a budes oni de aerosol).

In one or more embodiments, the glucocorticoid includes one or more of hydrocortisone, prednisone, prednisolone, methylprednisolone, dexamethasone and betamethasone.

In one or more embodiments, the Cystatin SN detection agent includes a quantitative detection agent of Cystatin SN protein.

In one or more embodiments, the Cystatin SN detection agent includes one or more of antibody or antibody fragment, agglutinin and aptamer capable of specifically binding the Cystatin SN protein.

A specific binding agent has an affinity of at least 10⁷ 1/mol for their corresponding target molecules. The specific binding agent preferably has an affinity of 10⁸ 1/mol, or more preferably 10⁹ 1/mol, for its target molecule. The skilled person would appreciate that using the term “specific” means that other biomolecules present in the sample do not significantly bind specific binding agent of the Cystatin SN protein. Preferably, the level of binding biomolecules other than the target molecule (Cystatin SN protein) results in a binding affinity which is at most 10% or less, only 5% or less, only 2% or less, or only 1% or less, respectively, of affinity to the target molecule. Preferred specific binding agents will simultaneously meet the minimum criteria for affinity and specificity in the above.

In the above, the Cystatin SN detection agent is preferably selected from an antibody or an antibody fragment, which may be packaged and exist in the form of an ELISA detection reagent, an antibody chip, an immunodetection kit.

In one or more embodiments, the Cystatin SN detection agent is used to detect Cystatin SN mRNA.

The term “for detecting Cystatin SN mRNA” should not to be understood in the present disclosure as merely a detection agent for Cystatin SN mRNA, but should include other detection reagents that may reflect the expression level of Cystatin SN mRNA known by a person skilled in the art. For example, the expression level of Cystatin SN mRNA may be detected indirectly by quantitatively detecting cDNA obtained by reverse transcription of Cystatin SN mRNA, or a polypeptide fragment obtained by transcription thereof

In one or more embodiments, the Cystatin SN detection agent includes a reagent suitable for at least one of the following methods:

A fluorescent dye method, digital PCR, a resonant light scattering method, real-time quantitative PCR, and sequencing or biomass spectrometry.

In one or more embodiments, the Cystatin SN detection agent is a probe or a primer capable of specifically binding Cystatin SN mRNA or Cystatin SN cDNA.

In one or more embodiments, the probe or primer carries a detectable label.

In one or more embodiments, the Cystatin SN detection agent is a qRT-PCR primer of Cystatin SN mRNA, of which an upstream primer is represented by SEQ ID NO. 2, and a downstream primer is represented by SEQ ID NO. 3.

In one or more embodiments, the kit further includes a reference gene primer.

In one or more embodiments, the reference gene is GAPDH, tubulin or actin.

In one or more embodiments, the qRT-PCR primer of the GAPDH has an upstream primer represented by SEQ ID NO. 4, and a downstream primer represented by SEQ ID NO. 5.

According to an aspect of the present disclosure, the present disclosure further relates to use of Cystatin SN in predicting sensitivity of a patient with chronic rhinosinusitis with nasal polyps to glucocorticoid; specifically:

A method for predicting sensitivity of a patient with chronic rhinosinusitis with nasal polyps to glucocorticoid, which includes:

(a) measuring the expression level of Cystatin SN mRNA or protein in a sample, (b) evaluating the sensitivity of a patient with chronic rhinosinusitis with nasal polyps to glucocorticoid using a measurement result of step (a), wherein the increased expression level of Cystatin SN mRNA or protein is (one of) the indicators of having sensitivity.

In one or more embodiments, the method further includes detecting the percentage of eosinophils, such as peripheral blood eosinophils.

An ideal scene for diagnosis is a situation in which a single event or process will cause a variety of diseases. In all of other cases, correct diagnosis may be quite difficult, especially when the etiology of the disease cannot be fully understood. As would be apparent to those skilled, no diagnosis of biochemical markers is 100% specificity and 100% sensitivity for a given multifactorial disease. Determining whether the subject sample has sensitivity compared with a normal control sample may be performed using statistical methods generally known in the art, and confirmed using a confidence interval and/or a p value. In some solutions, the confidence interval is 90%, 95%, 97.5%, 98%, 99%, 99.5%, 99.9% or 99.99% and the p value is 0.1, 0.05, 0.025, 0.02, 0.01, 0.005, 0.001 or 0.0001.

The present disclosure provides a method for detecting chronic rhinosinusitis with nasal polyps subtype, including detecting an expression level of a CST1 gene in nasal brushing cells, for example, detecting the expression level of the CST1 gene in the nasal brushing cells using the kit in the present disclosure.

In one or more embodiments, the CST1 gene is used as a biomarker in detecting the chronic rhinosinusitis with nasal polyps subtype or predicting sensitivity of a patient with chronic rhinosinusitis with nasal polyps to glucocorticoid.

In one or more embodiments, the expression level of the CST1 gene in nasal brushing cells is detected by fluorescence PCR.

In one or more embodiments, the chronic rhinosinusitis with nasal polyps subtype is determined according to ΔCt (Ct(CST1)-Ct(GAPDH)), where Ct(CST1) is the Ct value of the CST1 gene, and Ct(GAPDH) is the Ct value of the reference gene GAPDH.

In one or more embodiments, the ΔCt being greater than or equal to 2.993 represents noneosinophilic chronic rhinosinusitis with nasal polyps, and the ΔCt being less than 2.993 represents eosinophilic chronic rhinosinusitis with nasal polyps.

In one or more embodiments, the chronic rhinosinusitis with nasal polyps subtype is noneosinophilic chronic rhinosinusitis with nasal polyps or eosinophilic chronic rhinosinusitis with nasal polyps.

The present disclosure provides use of Cystatin SN or a coding gene CST1 thereof as a biomarker in detecting chronic rhinosinusitis with nasal polyps subtype or predicting sensitivity of a patient with chronic rhinosinusitis with nasal polyps to glucocorticoid.

The present disclosure provides use of a CST1 gene as a biomarker in detecting chronic rhinosinusitis with nasal polyps subtype.

In one or more embodiments, the Cystatin SN is human Cystatin SN.

The present disclosure provides a method for detecting chronic rhinosinusitis with nasal polyps subtype or predicting sensitivity of a patient with chronic rhinosinusitis with nasal polyps to glucocorticoid, including detecting an amount of Cystatin SN or a coding gene CST1 thereof in a patient sample.

The present disclosure provides use of CST1 or a coding gene CST1 thereof as a biomarker in detecting chronic rhinosinusitis with nasal polyps subtype or predicting sensitivity of a patient with chronic rhinosinusitis with nasal polyps to glucocorticoid.

Advantages and positive effects of the present disclosure at least include:

1. the present disclosure provides a kit for detecting chronic rhinosinusitis with nasal polyps subtype. Through screening by proteomics and transcriptomics methods, the CST1 gene is adopted as a biomarker, and applied to the kit, so as to realize the method for detecting chronic rhinosinusitis with nasal polyps subtype with the kit, so that the finally obtained kit includes the specific primer of the CST1 gene. On the basis of having this specific primer, the kit of the present disclosure can rapidly identify nasal polyp subtype, and has higher accuracy compared with the conventional pathological detection method. This kit may perform mass and rapid detection simultaneously on the samples, thus saving the labor costs and medical costs. Moreover, the systematic kit has higher identification accuracy, may comprehensively reflect histopathological characteristics, thus solving the influence of human error in the prior art, and avoiding the drawback of misdiagnosis caused by the fact that the tissue section reflects local characteristics of the tissues. Rapidly, accurately, and comprehensively identifying the nasal polyp subtype with the kit is of vital importance to clinical diagnosis and treatment, so as to perform targeted treatment according to the inflammation subtype of nasal polyps as soon as possible, effectively guide the determination of the medication mode and operation mode for the patients with chronic rhinosinusitis with nasal polyps, accurately estimate the response to medication, and judge the prognosis effect.

2. the kit provided in the present disclosure can obtain the nasal polyp cells from the surface of nasal polyps in a manner of brushing or sticking for detection, so as to determine the chronic rhinosinusitis with nasal polyps subtype of the patient, avoid wounds to the patient, and improve the safety of patient examination, moreover, the operation is more convenient, and labor costs and medical costs are saved.

Compared with the prior art, the advantages and positive effects of the present disclosure lie in:

3. the method for detecting an expression level of a CST1 gene in nasal brushing cells provided in the present disclosure, taking the effectively screened CST1 gene as a biomarker, provides a method for detecting the expression level of the gene, realizes the calculation of the expression level of the CST1 gene in the nasal brushing cells, and can effectively acquire the expression level of the CST1 gene. The method provided is simple and fast, and has high sensitivity and good repeatability, thus being suitable for wide popularization and application.

4. in the method for detecting an expression level of a CST1 gene in nasal brushing cells provided in the present disclosure, CST1 is a member of cysteine protease inhibitor family, a type 2 cystatin, and abundantly exists in saliva, with a physiological function of inhibiting the destructive action of cysteine proteases such as papain on oral epithelial cells. In recent years, it has been found that CST1 is abnormally expressed in some tumors such as pancreatic cancer and breast cancer, and can exist as a biomarker of aging. The method for detecting an expression level of a CST1 gene in nasal brushing cells provided in the present disclosure can be used for detecting the expression condition of the CST1 in the nasal brushing cells.

5. according to the method for detecting an expression level of a CST1 gene in nasal brushing cells provided in the present disclosure, a relative quantification method of ΔCt or 2^{-ΔΔCt} method is adopted according to actual requirements, the reference gene with relatively constant expression level is selected, the quantity of the reference genes is used for standardization, the target gene expression level is calculated by measuring the difference between the Ct values of the target gene of the sample and the reference gene. The method is simple and fast, with high detection accuracy, may reduce the detection cost, save the detection time, and have advantages such as easy interpretation of result, thus greatly improving the experimental efficiency.

6. the method for detecting an expression level of a CST1 gene in nasal brushing cells provided in the present disclosure provides a foundation for a gene screening technology for detecting the chronic rhinosinusitis with nasal polyps subtype in the future, and provides a reliable basis for clinical guidance and medication, thus ensuring the feasibility of the kit for detecting the chronic rhinosinusitis with nasal polyps subtype in clinical application.

7. the present disclosure proposes for the first time that the Cystatin SN can be used for predicting the sensitivity of the patients with chronic rhinosinusitis with nasal polyps to glucocorticoid, and the detection method is simple and fast, and has good prediction performance, thus having good clinical application prospect.

Embodiments of the present disclosure will be described in detail below in combination with examples, while a person skilled in the art would understand that the following examples are merely used for illustrating the present disclosure, but should not be considered as limitation to the scope of the present disclosure. If no specific conditions are specified in the examples, they are carried out under normal conditions or conditions recommended by manufacturers. If manufacturers of reagents or apparatuses used are not specified, they are conventional products commercially available.

### EXAMPLE 1

A kit for detecting chronic rhinosinusitis with nasal polyps subtype, including following reagents:

A reagent for extracting RNA from nasal polyp tissues: 20 mL of an RNA extraction solution Trizol or RNAiso Blood or RNAiso Plus or other substances containing phenol, guanidinium isothiocyanate, 8-hydroxyquinoline, guanidinium isothiocyanate or (3-mercaptoethanol, which can rapidly break off cells and inhibit substances of nuclease released from cells; 2 mL of chloroform; 20 mL of isopropanol; 40 mL of 6590% ethanol; and 5 mL of RNase-free and DNase-free water;

A reagent for reverse transcription of the extracted RNA to cDNA: 40 μL of a reverse transcription mixed solution (containing enzyme required for reverse transcription, an RNase inhibitor, a random 6-nucleotide primer, polythymine, T-repeat oligonucleotide, triphosphate deoxyribonucleotide mixture, a buffer solution, etc.), and 160 μL of RNase-free and DNase-free water, wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 25 uL of a premix (containing enzyme required for PCR and a buffer solution), 0˜50 uL of double distilled water (replenishing the system to 50 μL with water according to total volume), 0˜2 μL of dye (for fluorescence compensation and correction of machine), 100 μM upstream primer of the CST1 gene,100 μM downstream primer of the CST1 gene, 100 μM upstream primer of the reference gene, 100 μM downstream primer of the reference gene, 10 μg of positive control, and 10 μg of negative control, wherein the positive control is a plasmid containing CST1, and the negative control is an empty plasmid (plasmid vector).

### EXAMPLE 2

A kit for detecting chronic rhinosinusitis with nasal polyps subtype, including following reagents:

A reagent for extracting RNA from nasal polyp tissues: 1 mL of an RNA extraction solution Trizol or RNAiso Blood or RNAiso Plus or other substances containing phenol, guanidinium isothiocyanate, 8-hydroxyquinoline, guanidinium isothiocyanate or (3-mercaptoethanol, which can rapidly break off cells and inhibit substances of nuclease released from cells; 0.2 mL of chloroform; 0.2 mL of isopropanol; 0.2 mL of 6590% ethanol; and 0.05 mL of RNase-free and DNase-free water;

A reagent for reverse transcription of the extracted RNA to cDNA: 2 μof a reverse transcription mixed solution (containing enzyme required for reverse transcription, an RNase inhibitor, a random 6-nucleotide primer, polythymine, T-repeat oligonucleotide, triphosphate deoxyribonucleotide mixture, a buffer solution, etc.), and 7 μof RNase-free and DNase-free water, wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 5 μof a premix (containing enzyme required for PCR and a buffer solution), 0˜10 μL of double distilled water (replenishing the system to 10 μL with water according to total volume), 0˜2 pL of dye (for fluorescence compensation and correction of machine), 50 μM upstream primer of the CST1 gene, 50 μM downstream primer of the CST1 gene, 50 μM upstream primer of the reference gene, 50 μM downstream primer of the reference gene, 5 μg of positive control, and 5 μg of negative control, wherein the positive control is a plasmid containing CST1, and the negative control is an empty plasmid (plasmid vector).

### EXAMPLE 3

A kit for detecting chronic rhinosinusitis with nasal polyps subtype, including following reagents:

A reagent for extracting RNA from nasal polyp tissues: 0.1 mL of an RNA extraction solution Trizol or RNAiso Blood or RNAiso Plus or other substances containing phenol, guanidinium isothiocyanate, 8-hydroxyquinoline, guanidinium isothiocyanate or (3-mercaptoethanol, which can rapidly break off cells and inhibit substances of nuclease released from cells; 0.05 mL of chloroform; 0.015 mL of isopropanol; 0.0075 mL of 6590% ethanol; and 0.01 mL RNase-free and DNase-free water;

A reagent for making total RNA to undergo reverse transcription to cDNA: 1 μof a reverse transcription mixed solution (containing enzyme required for reverse transcription, an RNase inhibitor, a random 6-nucleotide primer, polythymine, T-repeat oligonucleotide, triphosphate deoxyribonucleotide mixture, a buffer solution, etc.), and 0˜10 pL of RNase-free and DNase-free water, wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 1 μof a premix (containing enzyme required for PCR and a buffer solution), 0˜10 μL of double distilled water (replenishing the system to 10 μL with water according to total volume), 0˜2 μL of dye (for fluorescence compensation and correction of machine), 1 μM upstream primer of the CST1 gene, 1 μM downstream primer of the CST1 gene, 1 μM upstream primer of the reference gene, 1 μM downstream primer of the reference gene, 1 μg of positive control, and 1 μg of negative control, wherein the positive control is a plasmid containing CST1, and the negative control is an empty plasmid (plasmid vector).

### EXAMPLE 4

A kit for detecting chronic rhinosinusitis with nasal polyps subtype, including following reagents and tool:

A reagent for extracting RNA from nasal polyp tissues: 100 μL of a cell lysis solution (an RL buffer solution added with of 50x dithiothreitol (DTT)), a genomic DNA adsorption column for removing genomic DNA, a collection tube for collecting solution after removal of genomic DNA, an RNA purification column for enriching RNA, 0.1 mL of a first buffer solution for removing impurities of the purification column adsorbed with RNA, a second buffer solution for removing impurities and salt in RNA solution, a centrifuge tube for collecting RNA, and 0.01 mL of RNase-free and DNase-free water for dissolving RNA;

A reagent for making total RNA to undergo reverse transcription to cDNA: 1 μof a reverse transcription mixed solution (containing enzyme required for reverse transcription, an RNase inhibitor, a random 6-nucleotide primers, polythymine, T-repeat oligonucleotide, triphosphate deoxyribonucleotide mixture, a buffer solution, etc.), and 0˜10 pL of RNase-free and DNase-free water, wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 25 of a premix (containing enzyme required for PCR and a buffer solution), 0˜10 μof double distilled water (replenishing the system to 10 μL with water according to total volume), 0˜2 μL of dye (for fluorescence compensation and correction of machine), 0.01 μM upstream primer of the CST1 gene, 0.01 μM downstream primer of the CST1 gene, 0.01 μM upstream primer of the reference gene, 0.01 pM downstream primer of the reference gene, 1 pg of positive control, and 1 μg of negative control, wherein the positive control is a plasmid containing CST1, and the negative control is an empty plasmid (plasmid vector).

### EXAMPLE 5

A kit for detecting chronic rhinosinusitis with nasal polyps subtype, including following reagents and tool:

A reagent for extracting RNA from nasal polyp tissues: 0.3 mL of a cell lysis solution (an RL buffer solution added with 50x dithiothreitol (DTT)), an RNA purification column for enriching RNA, 0.5 mL of a first buffer solution for removing impurities of the purification column adsorbed with RNA, 0.6 mL of a second buffer solution for removing impurities and salt in RNA solution, 4 μof a recombinant DNase for removing the genomic DNA, 5 μof a DNase buffer solution for removing the genomic DNA, 41 μL, of RNase-free double distilled water, a centrifuge tube for collecting RNA, and 0.05 mL of RNase-free and DNase-free water for dissolving RNA;

A reagent for making total RNA to undergo reverse transcription to cDNA: 2 μof a reverse transcription mixed solution (containing enzyme required for reverse transcription, an RNase inhibitor, a random 6-nucleotide primers, polythymine, T-repeat oligonucleotide, triphosphate deoxyribonucleotide mixture, a buffer solution, etc.), and 0˜10 pt of RNase-free and DNase-free water, wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 5 μof a premix (containing enzyme required for PCR and a buffer solution), 0˜10 μL of double distilled water (replenishing the system to 10 μL with water according to total volume), 0˜2 μL of dye (for fluorescence compensation and correction of machine), 10 pmol/L upstream primer of the CST1 gene, 10 pmol/L downstream primer of the CST1 gene, 10 pmol/L upstream primer of reference gene, 10 pmol/L downstream primer of the reference gene, 1 μg of positive control, and 1 μg of negative control, wherein the positive control is a plasmid containing CST1, and the negative control is an empty plasmid (plasmid vector).

### EXAMPLE 6

A kit for detecting chronic rhinosinusitis with nasal polyps subtype, including following reagents and tool:

A reagent for extracting RNA from nasal polyp tissues: 2 mL of a cell lysis solution (an RL buffer solution added with 50x dithiothreitol (DTT)), a genomic DNA adsorption column for removing genomic DNA, a collection tube for collecting solution after removal of genomic DNA, an RNA purification column for enriching RNA, 0.7 mL of a first buffer solution for removing impurities of the purification column adsorbed with RNA, 0.7 mL of a second buffer solution for removing impurities and salt in RNA solution, a centrifuge tube for collecting RNA, and 1 mL of RNase-free and DNase-free water for dissolving RNA;

A reagent for making total RNA to undergo reverse transcription to cDNA: 2 μof a reverse transcription mixed solution (containing enzyme required for reverse transcription, RNase inhibitor (RNA enzyme inhibitor), a random 6-nucleotide primers, polythymine, T-repeat oligonucleotide, triphosphate deoxyribonucleotide mixture, a buffer solution, etc.), and 0˜8 μL of RNase-free and DNase-free water (replenishing the system to 8 pL with water according to RNA amount), wherein the RNase-free and DNase-free water is used for replenishing the system, dissolving and diluting RNA; and

A reagent for performing real-time quantitative PCR reaction on the CST1 gene and the reference gene in the cDNA by adopting quantitative polymerase chain reaction: 5 μof a premix (containing enzyme required for PCR and a buffer solution), 0˜10 μL of double distilled water (replenishing the system to 10 μL with water according to total volume), 0˜2 μL of dye (for fluorescence compensation and correction of machine), 1 μM upstream primer of the CST1 gene, 1 μM downstream primer of the CST1 gene, 1 μM upstream primer of the reference gene, 1 μM downstream primer of the reference gene, 1 μg of positive control, and 1 μg of negative control.

In the above Examples 4˜6, the first buffer solution used, RWA buffer, is manufactured by Takara, Article No. 9767; and the second buffer solution, RWB buffer, is manufactured by Takara, Article No. 9767.

All of the kits provided in Examples 1˜6 of the present disclosure can realize the detection of chronic rhinosinusitis with nasal polyps subtype, and now experiments for detecting specific effects of the kit for detecting chronic rhinosinusitis with nasal polyps subtype are provided as follows.

Experiments for Effects of Detecting Nasal Polyp Subtype

### EXPERIMENTAL EXAMPLE 1

1. Experimental Method:

(1) Collecting and processing samples:

78 CRSwNP patients were randomly selected and rinsed the nasal cavities with physiological saline, and then the nasal polyps were taken under a nasal endoscope. The nasal polyps were cut into tissues with a diameter of about 0.5 cm, soaked in RNA stabilization and storage solution (RNAlater), stored at 4° C. for a short period, and then transferred to a temperature below −20° C. for long-term storage.

(2) Extracting RNA:

Step 1: weighing the tissues soaked in the RNA stabilization and storage solution (RNAlater), weighing about 0.01 g of the tissues into a centrifuge tube equipped with magnetic beads, placing the centrifuge tube in liquid nitrogen, and milling (3000 r, 5 min) (or manually grinding) the resultant on a homogenizer, to a test tube with tissue cells adding 1 mL of Trizol to dissolve the tissue cells, collecting the resultant into the centrifuge tube, sufficiently shaking the centrifuge tube, and standing at room temperature for 5 minutes; then adding 200 nt of the chloroform (trichloromethane) in the above reagent for extracting RNA, vigorously shaking and mixing the resultant uniformly, and standing at room temperature for 5 minutes;

Step 2: centrifuging the resultant at 12,000 r/min at 4° C. for 15 minutes;

Step 3: taking a supernatant, actually in a volume of about 200 nt, adding the supernatant to the centrifuge tube, adding isopropanol in an equal volume (about 200 !IL) in the above reagent for extracting RNA, after mixing them uniformly, standing for 10 minutes, centrifuging the resultant at 12000 r/min at 4° C. for 15 minutes, discarding the supernatant, and retaining a precipitate;

Step 4: adding about 200 !IL (in a volume equal to that of isopropanol) of 75% ethanol (a mixture of about 150 nt of anhydrous ethanol and 50 nt of DNase-free and RNase-free water) in the above reagent for extracting RNA to clean the precipitate, mixing them uniformly, centrifuging the resultant at 7500 r/min at 4° C. for 15 minutes, discarding a supernatant, and retaining the precipitate;

Step 5: capping the centrifuge tube tightly, and centrifuging the resultant at 7500 r/min at 4° C. for 2 minutes;

Step 6: uncapping the centrifuge tube, discarding the supernatant, and standing in a fuming cupboard for 15 minutes;

Step 7: adding 0.02 mL of the RNase-free and DNase-free water in the above reagent for extracting RNA to dissolve the precipitate; and

Step 8: measuring RNA concentration using a spectrophotometer, wherein a ratio of OD260/OD280 was 1.7˜2.1.

(3) Preparing cDNA by reverse transcription: formulating a reverse transcription (RT) reaction solution on ice, specifically including the following reagents: 2 μof a reverse transcription mixed solution (containing the enzyme required for reverse transcription, an RNase inhibitor, a random 6-nucleotide primer, polythymine, T-repeat oligonucleotide, triphosphate deoxyribonucleotide mixture, a buffer solution, etc.). total RNA not exceeding 500 ng or not exceeding 8 μ, 0˜8 μL of RNase-free and DNase-free water (replenishing the system to 8 μwith water according to RNA amount); adding the extracted total RNA and the above reverse transcription reaction solution to the reaction system to perform reverse transcription reaction to obtain a cDNA template, wherein the reaction system may be correspondingly amplified according to requirements, and a maximum of 500 ng of total RNA may be used in a 10 μL reaction system;

Reverse transcription reaction condition is as follows:

37° C. for 15 min (reverse transcription reaction)

84° C. for 5 seconds (inactivation reaction of reverse transcriptase)

placing a product at 4° C.

(4) Preparing a real-time quantitative PCR reaction solution: including 5 μof a premix (containing enzyme required for PCR and a buffer solution), 0.2 4, of a machine fluorescence compensation and correction agent, 1 ng/μL of cDNA or a positive control or negative control, 0.5 μL of an upstream primer of the CST1 gene, 0.5 μL of a downstream primer of the CST1 gene, 0.5 μL of an upstream primer of the reference gene, 0.5 μL of a downstream primer of the reference gene and 2.8 μL of double distilled water;

(5) Real-time quantitative PCR detection:

Reaction conditions:

Stage 1: pre-denaturation at 95° C. for 30 seconds.

Stage 2: PCR reaction at 95° C. for 15 seconds; and annealing and extension at 60° C. for 1 minute, 40 cycles in total, wherein in stage 2, in a melting curve: 60° C. is gradually increased to 95° C., at a rate of 0.1° C./s, and fluorescence was collected;

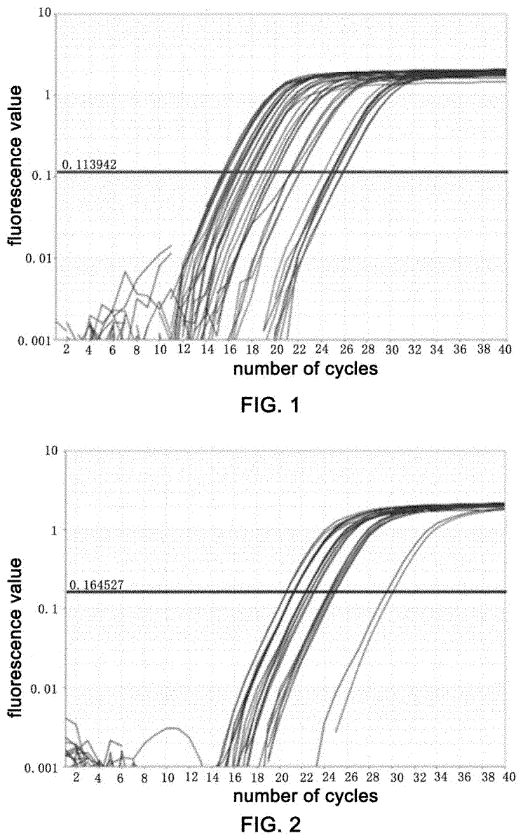

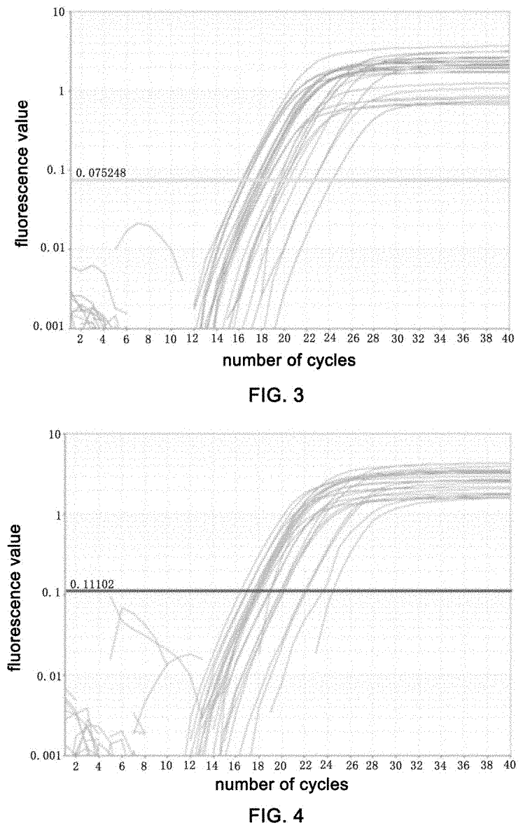

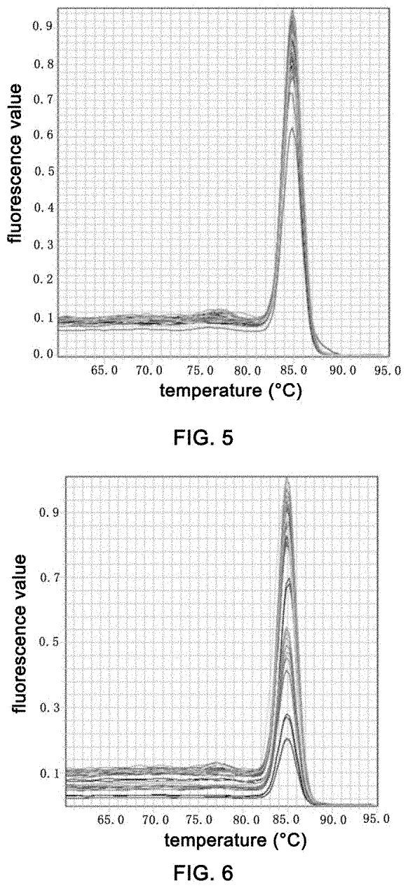

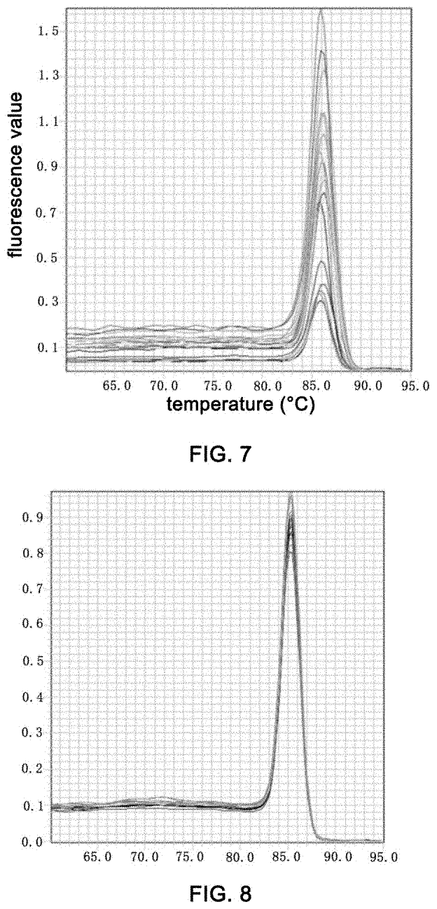

After the reaction was completed, a confirmed amplification curve of the real-time quantitative PCR is as shown in FIG. 1 to FIG. 4, and the melting curve is shown in FIG. 5 to FIG. 8. The Ct values of the CST1 and the GAPDH were read, analysis was performed using the ΔCt analysis method (the Ct value of the CST1 minus the Ct value of the GAPDH), and the GAPDH was used as the reference gene.

(6) Data analysis:

Step 1: judging the experimental quality control: the experiment was considered valid if a positive control Ct value is less than 20 and a negative control Ct value is greater than 38, otherwise the experiment was invalid;

Step 2: judging the typing: the Ct value of the target gene minus the Ct value of the reference gene (ΔCt value), wherein according to the ROC curve, an optimal threshold value of the ΔCt value is 2.993, and if the ΔCt value is greater than or equal to 2.993, it is noneosinophilic chronic rhinosinusitis with nasal polyps; and if the ΔCt value is less than 2.993, it is typical eosinophilic chronic rhinosinusitis with nasal polyps.

2. Experimental results: nasal polyp subtype detection results of the 78 selected patients based on the kit provided in the above are as shown in Table 1:

TABLE 1
Nasal Polyp Subtype Detection Results by Kit Provided in the Present
Disclosure and Histopathologicallv Acquired Nasal Polyp Tissues

| Subject | ΔCt | Result Judged According | |
|---|---|---|---|
| No. | Value | to Threshold Value | Pathologic Result |
| 1 | 9.079 | noneosinophilic | noneosinophilic |
| 2 | 9.015 | noneosinophilic | noneosinophilic |
| 3 | 7.884 | noneosinophilic | noneosinophilic |
| 4 | 8.431 | noneosinophilic | noneosinophilic |
| 5 | 4.535 | noneosinophilic | noneosinophilic |
| 6 | 0.302 | eosinophilic | eosinophilic |
| 7 | 8.886 | noneosinophilic | noneosinophilic |
| 8 | −0.491 | eosinophilic | eosinophilic |
| 9 | 7.832 | noneosinophilic | noneosinophilic |
| 10 | 6.730 | noneosinophilic | noneosinophilic |
| 11 | 4.825 | noneosinophilic | noneosinophilic |
| 12 | 5.339 | noneosinophilic | noneosinophilic |
| 13 | 4.239 | noneosinophilic | noneosinophilic |
| 14 | 3.125 | noneosinophilic | noneosinophilic |
| 15 | 4.173 | noneosinophilic | noneosinophilic |
| 16 | 8.571 | noneosinophilic | noneosinophilic |
| 17 | 6.324 | noneosinophilic | noneosinophilic |
| 18 | 6.370 | noneosinophilic | noneosinophilic |
| 19 | 4.388 | noneosinophilic | noneosinophilic |
| 20 | 8.296 | noneosinophilic | noneosinophilic |
| 21 | 5.996 | noneosinophilic | noneosinophilic |
| 22 | 7.245 | noneosinophilic | noneosinophilic |
| 23 | 0.676 | eosinophilic | eosinophilic |
| 24 | 6.935 | noneosinophilic | noneosinophilic |
| 25 | 7.280 | noneosinophilic | noneosinophilic |
| 26 | 7.290 | noneosinophilic | noneosinophilic |
| 27 | 2.450 | eosinophilic | noneosinophilic |
| 28 | 0.408 | eosinophilic | eosinophilic |
| 29 | 3.108 | noneosinophilic | eosinophilic |
| 30 | 2.135 | eosinophilic | eosinophilic |
| 31 | 4.141 | noneosinophilic | eosinophilic |
| 32 | 1.183 | eosinophilic | eosinophilic |
| 33 | −1.702 | eosinophilic | eosinophilic |
| 34 | 3.512 | noneosinophilic | eosinophilic |
| 35 | −2.615 | eosinophilic | eosinophilic |
| 36 | 1.531 | eosinophilic | eosinophilic |
| 37 | 2.831 | eosinophilic | eosinophilic |
| 38 | 3.401 | noneosinophilic | eosinophilic |
| 39 | 3.521 | noneosinophilic | eosinophilic |
| 40 | 3.315 | noneosinophilic | eosinophilic |
| 41 | −1.545 | eosinophilic | eosinophilic |
| 42 | 2.999 | noneosinophilic | noneosinophilic |
| 43 | −1.565 | eosinophilic | eosinophilic |
| 44 | 0.722 | eosinophilic | eosinophilic |
| 45 | −0.603 | eosinophilic | eosinophilic |
| 46 | 1.616 | eosinophilic | eosinophilic |
| 47 | −1.542 | eosinophilic | eosinophilic |
| 48 | 3.839 | noneosinophilic | eosinophilic |
| 49 | 1.258 | eosinophilic | eosinophilic |
| 50 | 2.459 | eosinophilic | eosinophilic |
| 51 | 0.961 | eosinophilic | eosinophilic |
| 52 | −1.617 | eosinophilic | eosinophilic |
| 53 | −0.781 | eosinophilic | eosinophilic |
| 54 | 0.045 | eosinophilic | eosinophilic |
| 55 | −0.933 | eosinophilic | eosinophilic |
| 56 | −0.586 | eosinophilic | eosinophilic |
| 57 | −0.507 | eosinophilic | eosinophilic |
| 58 | 2.986 | eosinophilic | eosinophilic |
| 59 | −0.053 | eosinophilic | eosinophilic |
| 60 | 0.288 | eosinophilic | eosinophilic |
| 61 | 0.677 | eosinophilic | eosinophilic |
| 62 | −3.541 | eosinophilic | eosinophilic |
| 63 | −0.993 | eosinophilic | eosinophilic |
| 64 | 2.492 | eosinophilic | eosinophilic |
| 65 | 2.649 | eosinophilic | noneosinophilic |
| 66 | 0.083 | eosinophilic | eosinophilic |
| 67 | 3.276 | noneosinophilic | eosinophilic |
| 68 | 3.814 | noneosinophilic | noneosinophilic |
| 69 | −0.651 | eosinophilic | eosinophilic |
| 70 | 2.783 | eosinophilic | eosinophilic |
| 71 | −0.309 | eosinophilic | eosinophilic |
| 72 | −0.821 | eosinophilic | eosinophilic |
| 73 | −3.337 | eosinophilic | eosinophilic |
| 74 | −0.042 | eosinophilic | eosinophilic |
| 75 | 1.214 | eosinophilic | eosinophilic |
| 76 | 1.978 | eosinophilic | eosinophilic |
| 77 | 2.105 | eosinophilic | eosinophilic |
| 78 | −0.545 | eosinophilic | eosinophilic |

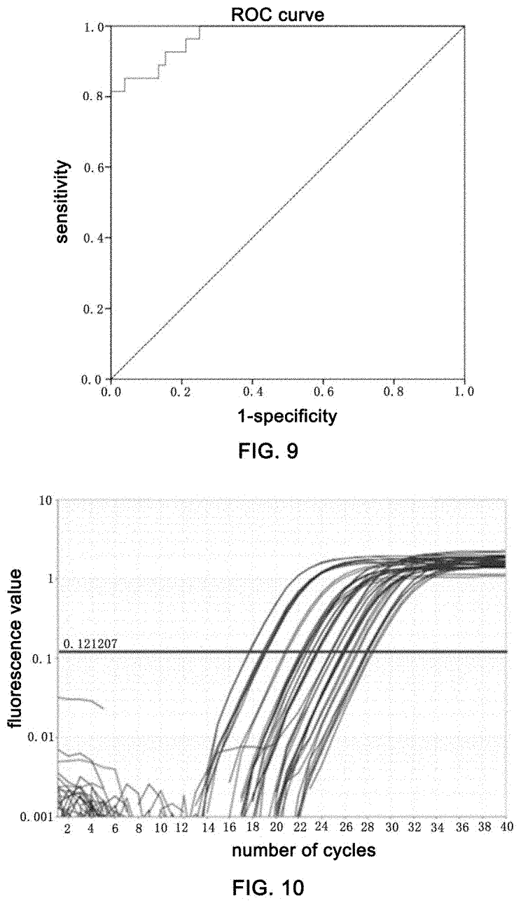

The ROC graph of the above 78 samples is shown in FIG. 9. Using the present kit to predict the typing of chronic rhinosinusitis with nasal polyps has the accuracy of 87.2%.

### EXPERIMENTAL EXAMPLE 2

1. Experimental Method:

(1) Collecting and processing samples:

After 47 CRSwNP patients were randomly selected and rinsed the nasal cavities with physiological saline, the surface of the nasal polyps were pressed by a brush (manufactured by Copan) under a nasal endoscope for 30 s, the brush was rotated for 3˜4 turns to brush the nasal polyp surface, the brush was placed in a lysis solution, and stored at 4° C. for a short period (not exceeding 24 hours), or transferred to a temperature below -20° C. for long-term storage.

(2) Extracting RNA:

Step 1: to a test tube with exfoliated cells adding 1 mL of Trizol to dissolve the cells, sufficiently shaking the test tube, standing at room temperature for 5 minutes, then adding 200 pi, of chloroform (trichloromethane), vigorously shaking and mixing the resultant uniformly, and standing at room temperature for 5 minutes;

Step 2: centrifuging the resultant at 12,000 r/min at 4° C. for 15 minutes;

Step 3: taking a supernatant, actually in a volume of about 200 μL, adding the supernatant to the centrifuge tube, adding isopropanol in a volume (about 200 μL) equal to that of the chloroform, after mixing them uniformly, standing for 10 minutes, centrifuging the resultant at 12000 r/min at 4° C. for 15 minutes, discarding the supernatant, and retaining a precipitate;

Step 4: adding about 200 μL (in a volume equal to that of isopropanol) of 75% ethanol (a mixture of about 150 μL of anhydrous ethanol and 50 μL of DNase-free and RNase-free water) in the above reagent for extracting RNA to clean the precipitate, mixing them uniformly, centrifuging the resultant at 7500 r/min at 4° C. for 15 minutes, discarding the supernatant, and retaining the precipitate;

Step 5: capping the centrifuge tube tightly, and centrifuging the resultant at 7500 r/min at 4° C. for 2 minutes;

Step 6: uncapping the centrifuge tube, discarding the supernatant, and standing in a fuming cupboard for 15 minutes;

Step 7: adding 0.02 mL of RNase-free and DNase-free water in the above reagent for extracting RNA to dissolve the precipitate; and

Step 8: measuring RNA concentration using a spectrophotometer, wherein it was better for the ratio of OD260/OD280 to be 1.72.1;

(3) Preparing cDNA by reverse transcription: the same as in Experimental Example 1;

(4) Preparing a real-time quantitative PCR reaction solution: the same as in Experimental Example 1;

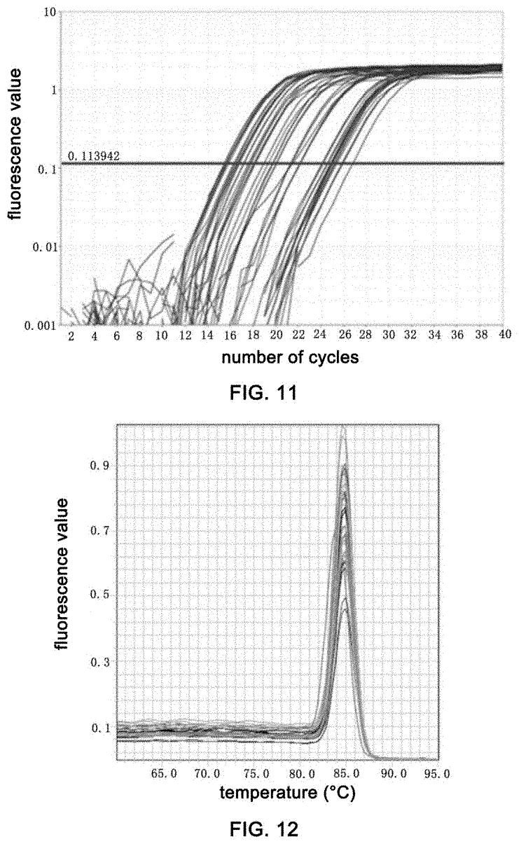

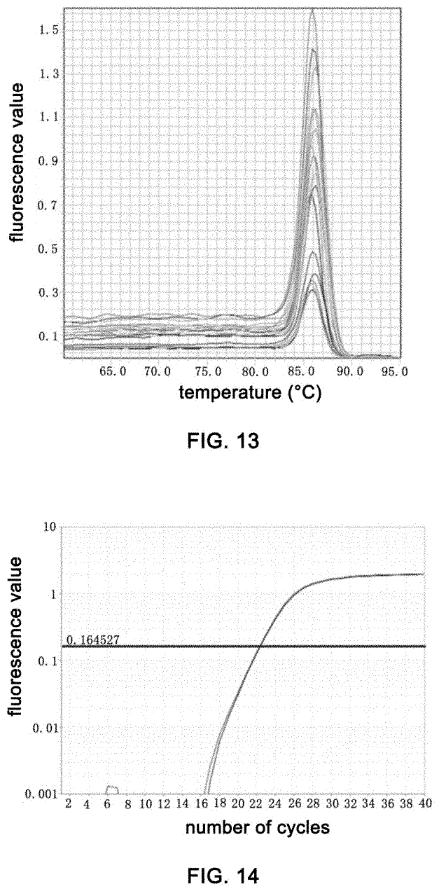

(5) Real-time quantitative PCR detection: the same as in Experimental Example 1; after the reaction was completed, a confirmed amplification curve of the real-time quantitative PCR is as shown in FIG. 10 and FIG. 11, and the melting curve is as shown in FIG. 12 and FIG. 13;

(6) Data analysis: the same as in Experimental Example 1.

2. Experimental results: calculation results are as shown in Table 2.

TABLE 2
Nasal Polyp Subtype Detection Results of Nasal Mucosa Exfoliated
Cells Obtained by Brushing the Surface of Nasal Polyps
with the Kit Provided in the Present Disclosure

| Subject | ΔCt | Pathologic Result Judged | |
|---|---|---|---|
| No. | Value | According to Threshold Value | Pathologic Result |
| 1 | −1.87 | eosinophilic | eosinophilic |
| 2 | 8.6 | noneosinophilic | noneosinophilic |
| 3 | 0.37 | eosinophilic | eosinophilic |
| 4 | 11.32 | noneosinophilic | noneosinophilic |
| 5 | 5.33 | noneosinophilic | noneosinophilic |
| 6 | 7.54 | noneosinophilic | noneosinophilic |
| 7 | 11.33 | noneosinophilic | noneosinophilic |
| 8 | 0.14 | eosinophilic | eosinophilic |
| 9 | 14.54 | noneosinophilic | noneosinophilic |
| 10 | 5.82 | noneosinophilic | noneosinophilic |
| 11 | 8.91 | noneosinophilic | noneosinophilic |
| 12 | 3.46 | noneosinophilic | noneosinophilic |
| 13 | −0.61 | eosinophilic | eosinophilic |
| 14 | 3.25 | noneosinophilic | eosinophilic |
| 15 | 0.66 | eosinophilic | eosinophilic |
| 16 | 0.46 | eosinophilic | eosinophilic |
| 17 | −1.93 | eosinophilic | eosinophilic |
| 18 | 3.45 | noneosinophilic | noneosinophilic |
| 19 | 3.19 | noneosinophilic | eosinophilic |
| 20 | 7.39 | noneosinophilic | noneosinophilic |
| 21 | 12.74 | noneosinophilic | noneosinophilic |
| 22 | −2.41 | eosinophilic | eosinophilic |
| 23 | 0.38 | eosinophilic | eosinophilic |
| 24 | 13.92 | noneosinophilic | noneosinophilic |
| 25 | 3.05 | noneosinophilic | noneosinophilic |
| 26 | 3.27 | noneosinophilic | noneosinophilic |
| 27 | 3.61 | noneosinophilic | eosinophilic |
| 28 | −0.77 | eosinophilic | eosinophilic |
| 29 | −2.14 | eosinophilic | eosinophilic |
| 30 | −1.24 | eosinophilic | eosinophilic |
| 31 | 0.85 | eosinophilic | eosinophilic |
| 32 | −3.54 | eosinophilic | eosinophilic |
| 33 | −0.59 | eosinophilic | eosinophilic |
| 34 | 1.9 | eosinophilic | eosinophilic |
| 35 | −0.54 | eosinophilic | eosinophilic |
| 36 | −0.02 | eosinophilic | eosinophilic |
| 37 | −0.96 | eosinophilic | eosinophilic |
| 38 | −0.36 | eosinophilic | eosinophilic |
| 39 | −2.73 | eosinophilic | eosinophilic |
| 40 | 1.92 | eosinophilic | eosinophilic |
| 41 | −1.01 | eosinophilic | eosinophilic |
| 42 | −1.75 | eosinophilic | eosinophilic |
| 43 | −1.35 | eosinophilic | eosinophilic |
| 44 | 2.42 | eosinophilic | eosinophilic |
| 45 | −1.3 | eosinophilic | eosinophilic |
| 46 | −0.99 | eosinophilic | eosinophilic |
| 47 | 8.68 | noneosinophilic | noneosinophilic |

From the data results provided in Table 2, it is obtained that the accuracy of the kit for detecting chronic rhinosinusitis with nasal polyps subtype in Experimental Example 2 of the present disclosure is 93.6%.

### EXPERIMENTAL EXAMPLE 3

1. Test Method:

(1) Collecting and processing samples:

After a patient rinsed the nasal cavity with physiological saline, the surface of the nasal polyps was pressed by a brush (manufactured by Copan) under a nasal endoscope for 30 s, the brush was rotated for 3˜4 turns to brush the nasal polyp surface, the brush was placed in a lysis solution, and stored at 4° C. for a short period (not exceeding 24 hours), or transferred to a temperature below −20° C. for long-term storage.

(2) Extracting RNA:

Step 1: placing a genomic DNA adsorption column (genomic DNA Eraser Spin Column) in a 2 mL collection tube (Collection Tube);

Step 2: transferring the lysis solution containing exfoliated cells (cell lysis solution) into the genomic DNA adsorption column;

Step 3: centrifuging the resultant at 12,000 r/min for 1 minute;

Step 4: discarding the genomic DNA adsorption column, and retaining a filtrate in the 2 mL collection tube;

Step 5: adding 300 uL of 70% ethanol (precipitate may appear at this time) to the above step 4, and mixing the solution uniformly using a pipette gun;

Step 6: immediately transferring the mixed solution (containing the precipitate) into an RNA purification column (RNA Spin Column) (containing the 2 mL collection tube);

Step 7: centrifuging the resultant at 12,000 r/min for 1 minute, discarding the filtrate, and placing the RNA purification column back into the 2 mL collection tube;

Step 8: adding 500 uL of a first buffer solution (Buffer RWA) to the RNA purification column, centrifuging the resultant at 12,000 r/min for 30 seconds, and discarding the filtrate;

Step 9: adding 600 uL of a second buffer solution (Buffer RWB) to the RNA purification column, centrifuging the resultant at 12,000 r/min for 30 seconds, and discarding the filtrate;

Step 10: positioning the RNA purification column on a 1.5 mL RNase-free collection tube (RNase Free Collection Tube), adding 50 uL of RNase-free distilled water (RNase Free dH2O) or 0.1% diethyl pyrocarbonate (DEPC) treated water at the center of the RNA purification column membrane, and standing at room temperature for 5 minutes;

Step 11: centrifuging the resultant at 12,000 r/min, and eluting with RNase-free and DNase-free water for 2 minutes; and

Step 12: measuring RNA concentration using a spectrophotometer, wherein the ratio of OD260/OD280 was 2.0.

(3) Preparing cDNA by reverse transcription: the same as in Experimental Example 1;

(4) Preparing a real-time quantitative PCR reaction solution:

Step 1: preparing 45 uL of SYBR Green 1 premix; mixing the SYBR Green 1 premix with 1.8 uL of ROX uniformly, dividing the resultant into 3 parts: A. 11.7 uL, B. 11.7 ut, and C. 23.4 uL, respectively, adding 1 ng/μL positive control to A to obtain a solution A, adding 1 ng/μL negative control to B to obtain a solution B, and adding 2 ng of the obtained cDNA to C to obtain a solution C, respectively (wherein the SYBR Green 1 premix and the ROX are both products of Takara, with Article No. RR820A);

Step 2: configuring 8 groups of parallel wells;

Parallel wells in groups 1 and 2: the solution A, the specific primer of the CST1 gene, and 3.8 μL of sterilized double distilled water;

Parallel wells in groups 3 and 4: the solution B, the specific primer of the CST1 gene, and 3.8 μL of sterilized double distilled water;

Parallel wells in groups 5 and 6: the solution C, the specific primer of the CST1 gene, and 3.8 μL of sterilized double distilled water;

Parallel wells in groups 7 and 8: the solution C, the specific primer of the GAPDH gene, and 3.8 μL of sterilized double distilled water;

Step 3: sealing the plate with a transparent adhesive film, centrifuging the resultant, and performing a PCR operation;

Step 4: two-step PCR amplification standard procedure:

(5) Real-time quantitative PCR detection: Reaction condition:

Stage 1: pre-denaturation at 95° C. for 30 seconds.

Stage 2: PCR reaction at 95° C. for 15 seconds; and annealing and extension at 60° C. for 1 minute, 40 cycles in total.

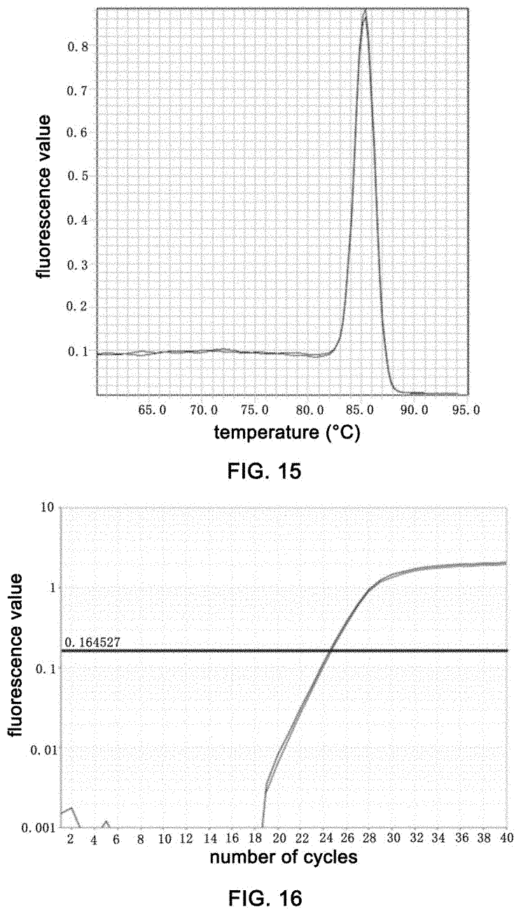

After the reaction was completed, a confirmed amplification curve of the real-time quantitative PCR is as shown in FIG. 14, and a melting curve is as shown in FIG. 15. The Ct values of the CST1 and the GAPDH were read, analysis was performed using the ΔCt analysis method (the Ct value of the CST1 minus the Ct value of the GAPDH), and the GAPDH was used as the reference gene.

(6) Data analysis: the same as in Experimental Example 1;

2. Experimental results: mean Ct of the positive control wells is 16.2, mean Ct of the negative control wells is 39.7, mean Ct of the sample CST1 is 22.5, and mean Ct of the sample GAPDH is 16.4, then the difference value is 22.5˜16.4, namely, 6.1, greater than 2.993, thus indicating noneosinophilic chronic rhinosinusitis with nasal polyps.

### EXPERIMENTAL EXAMPLE 4

All of the kits provided in Examples 1˜6 of the present disclosure can realize the detection for chronic rhinosinusitis with nasal polyps subtype. Now Example 5 is taken as an example below to carry out an experiment for detecting the effect of the kit for detecting chronic rhinosinusitis with nasal polyps subtype.

(1) Collecting and processing samples:

After a patient rinsed the nasal cavity with physiological saline, the nasal polyps were taken under a nasal endoscope. The polyps were cut into tissues with a diameter of about 0.5 cm, soaked in RNA stabilization and storage solution (RNAlater), stored at 4° C. for a short period, and then transferred to a temperature below -20° C. for long-term storage.

(2) Extracting RNA:

Step 1: weighing the tissues soaked in the RNA stabilization and storage solution (RNAlater), weighing about 0.01 g of the tissues into a centrifuge tube equipped with magnetic beads, placing the centrifuge tube in liquid nitrogen, milling (3000 r, 5 min) (or manually grinding) the resultant on a homogenizer; adding 0.3 mL of a cell lysis solution, and centrifuging the resultant at 12,000 r/min for 15 minutes;

Step 2: pipetting a supernatant, adding 70% ethanol (70% anhydrous ethanol and 30% DEPC or RNase-free and DNase-free water) in a volume equal to that of the supernatant, and mixing the solution uniformly using a pipette gun;

Step 3: immediately transferring the mixed solution (containing the precipitate) into an RNA purification column (containing a 2 mL collection tube);

Step 4: centrifuging the resultant at 12,000 r/min for 1 minute, discarding the filtrate, and placing the RNA purification column back into the 2 mL collection tube;

Step 5: adding 500 !IL of a first buffer solution (Buffer RWA) to the RNA purification column, centrifuging the resultant at 12,000 r/min for 30 seconds, and discarding the filtrate;

Step 6: adding 600 !IL of a second buffer solution (Buffer RWB) to the RNA purification column, centrifuging the resultant at 12,000 r/min for 30 seconds, and discarding the filtrate;

Step 7: preparing a DNase I reaction solution: pipetting 5 μof 10x DNase I buffer solution, 4 μof recombinant DNase I (RNase-free, 5U/μL), and 41 !IL of RNase-free double distilled water into a new 1.5 mL tube (RNase-free) to be mixed uniformly;

Step 8: adding 50 !IL of the DNase I reaction solution into the center of the RNA purification column membrane, and standing at room temperature for 15 minutes;

Step 9: adding 350 !IL of a second buffer solution into the center of the RNA purification column membrane, centrifuging the resultant at 12,000 r/min for 30 seconds, and discarding a filtrate;

Step 10: repeating step 6;

Step 11: repositioning the RNA purification column on the 2 mL collection tube, and centrifuging the resultant at 12,000 r/min for 2 minutes;

Step 12: positioning the RNA purification column on 1.5 mL RNase-free collection tube (RNase Free Collection Tube), adding 50 uL of RNase-free distilled water (RNase Free dH2O) or 0.1% diethyl pyrocarbonate (DEPC) treated water at the center of the RNA purification column membrane, and standing at room temperature for 5 minutes;

Step 13: centrifuging the resultant at 12,000 r/min, and eluting with RNase-free and DNase-free water for 2 minutes; and

Step 14: measuring RNA concentration using a spectrophotometer, wherein a ratio of OD260/OD280 was 2.0.

(3). Preparing cDNA by reverse transcription: the same as in Experimental Example 1;

(4) Preparing a real-time quantitative PCR reaction solution: the same as in Experimental Example 3;

(5) Real-time quantitative PCR detection:

The reaction condition adopts a three-step PCR amplification standard procedure:

Stage 1: pre-denaturation at 95° C. for 2 minutes;

Stage 2: PCR reaction at 95° C. for 1 minute, 55° C. for 1 minute, and 72° C. for 1 minute, 40 cycles in total, and finally annealing and extension at 72° C. for 7 minutes;

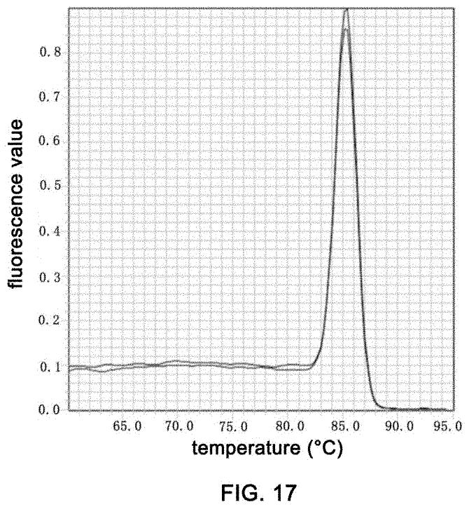

After the reaction was completed, a confirmed amplification curve of the real-time quantitative PCR is as shown in FIG. 16, and a melting curve is as shown in FIG. 17. The Ct values of the CST1 and the GAPDH were read, analysis was performed using the ΔCt analysis method (the Ct value of the CST1 minus the Ct value of the GAPDH), and the GAPDH was used as the reference gene.

(6) Data analysis: the same as in Experimental Example 1;

2. Experimental results: mean Ct of the positive control wells is 16.7, mean Ct of the negative control wells is 38.4, mean Ct of the sample CST1 is 24.7, and mean Ct of the sample GAPDH is 19.4, then the difference value is 5.3, greater than 2.993, thus indicating noneosinophilic chronic rhinosinusitis with nasal polyps.

To sum up, the present disclosure provides a kit for detecting chronic rhinosinusitis with nasal polyps subtype, the CST1 gene is screened as a biomarker, and is applied to the kit, so as to realize the method for detecting chronic rhinosinusitis with nasal polyps subtype with the kit. The kit can rapidly, accurately, and comprehensively identify the nasal polyp subtype of the patients, so as to perform targeted treatment according to the inflammation subtype of nasal polyps as soon as possible, effectively guide the determination of the medication mode and operation mode for the patients with chronic rhinosinusitis with nasal polyps, accurately estimate the response to medication, and judge the prognosis effect.

### EXAMPLE 7

Collecting and processing samples:

After a patient rinsed the nasal cavity with physiological saline, the surface of the nasal polyps were pressed by a brush (manufactured by Copan) under a nasal endoscope for 30 s, the brush was rotated for 3˜4 turns to brush the polyp surface, the brush was placed in a lysis solution, and stored at 4° C. for a short period (not exceeding 24 hours), or transferred to a temperature below −20° C. for long-term storage.

A method for detecting an expression level of a CST1 gene in nasal brushing cells, including following steps:

Step I: extracting RNA from nasal brushing cells;

Step 1: putting a genomic DNA adsorption column into a 2 mL collection tube, dissolving the nasal brushing cells in 300 μL of a cell lysis solution, then adding the resultant into the genomic DNA adsorption column, taking a filtrate, adding 70% ethanol in the same volume to the filtrate, mixing them uniformly, adding the resultant into an RNA purification column, centrifuging the resultant at 12000 r/min for 1 min, removing a filtrate, and placing the RNA purification column into a 2 mL collection tube;

Step 2: adding 500 μL of a first buffer solution to the RNA purification column obtained in step 1, centrifuging the resultant at 12000 r/min for 30 s, and removing a first filtrate; adding 600 μL of a second buffer solution to the RNA purification column continuously, centrifuging the resultant at 12000 r/min for 30 s, and removing a second filtrate;

Step 3: positioning the RNA purification column from which the second filtrate was removed in step 2 in a 1.5 mL RNase-free collection pipe, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA, with measuring the ratio of OD260/OD280 of the RNA solution to be 2.0 using a spectrophotometer;

Step II: preparing cDNA by reverse transcription, including following steps: taking 2 μof the reverse transcription mixed solution, 0˜8 μL of the RNase-free distilled water (replenishing the system to 8 μwith water according to the RNA amount), and total RNA in a total amount not exceeding 500 ng or a volume not exceeding 8 μ, wherein the RNase-free distilled water was used to replenish the system to 10 0_{—4} gently mixing them uniformly and then carrying out reverse transcription reaction, under the following condition: carrying out the reverse transcription reaction under a condition of 37° C. for 15 minutes; carrying out inactivation reaction of reverse transcriptase under a condition of 85° C. for 5 seconds; and placing a product at 4° C.

Step III: Real-time quantitative PCR amplification detection, including following steps:

Step 1: preparing a real-time quantitative PCR reaction solution: including 1 μof a PCR premix, 0˜10 μL of double distilled water (replenishing the system to 10 μL with water according to the total volume), 0.2 μL of machine fluorescence compensation and correction agent, 1 μM upstream primer of the CST1 gene, 1 μM downstream primer of the CST1 gene, 1 04 upstream primer of the reference gene, 1 μM downstream primer of the reference gene, 0.01 μL of the cDNA, 1 μg of positive control, and 1 μg of negative control, wherein the positive control is a plasmid containing CST1, and the negative control is an empty plasmid (plasmid vector);

Step 2: adopting a two-step PCR amplification standard procedure, wherein the reaction condition of the two-step PCR amplification standard procedure includes the following steps: stage 1: pre-denaturation under a condition of 95° C. for 30 seconds; stage 2: PCR reaction under a condition of 95° C. for 15 seconds, and under a condition of 60° C. for 60 seconds, annealing and extension, 40 cycles in total as such.

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid; and

By comparing the difference between expression levels of the CST1 and the reference gene using the ΔCt method, it is obtained that a mean CT value of the CST1 is 20.1, and a mean CT value of GAPDH is 18.9, then the ACT value is 1.2.

### EXAMPLE 8:

Collecting and processing samples:

After a patient rinsed the nasal cavity with physiological saline, the surface of the nasal polyps were pressed by a brush (manufactured by Copan) under a nasal endoscope for 30 s, the brush was rotated for 3˜4 turns to brush the polyp surface, the brush was placed in a lysis solution, and stored at 4° C. for a short period (not exceeding 24 hours), or transferred to a temperature below -20° C. for long-term storage.

A method for detecting an expression level of a CST1 gene in nasal brushing cells, including following steps:

Step I: extracting RNA from nasal brushing cells;

Step 1: putting a genomic DNA adsorption column into a 2 mL collection tube, dissolving the nasal brushing cells in 100 n.L of a cell lysis solution, then adding the resultant into the genomic DNA adsorption column, centrifuging the resultant at 12000r/min for 60 s, taking a filtrate, adding 70% ethanol in the same volume to the filtrate, mixing them uniformly, adding the resultant into an RNA purification column, centrifuging the resultant at 12000 r/min for 1 min, removing a filtrate, and placing the RNA purification column into a 2 mL collection tube;

Step 2: adding 300 nL of a first buffer solution to the RNA purification column obtained in step 1, centrifuging the resultant at 12000 r/min for 30 s, and removing a first filtrate; adding 400 n.L of a second buffer solution to the RNA purification column continuously, centrifuging the resultant at 12000 r/min for 30 s, and removing a second filtrate;

Step 3: placing the RNA purification column from which the second filtrate was removed in step 2 in a 1.5 mL RNase-free collection pipe, adding 50 nL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA, with measuring the ratio of OD260/OD280 of the RNA solution to be 2.0 using a spectrophotometer;

Step II: preparing cDNA by reverse transcription, including following steps: taking 1 μof the reverse transcription mixed solution, 0˜10 n.L of the RNase-free distilled water, and total RNA in a total amount not exceeding 500 ng or a volume not exceeding 8 μ, wherein the RNase-free distilled water was used to replenish the system to 10 nL; gently mixing them uniformly and then carrying out reverse transcription reaction, under the following condition: carrying out the reverse transcription reaction under a condition of 37° C. for 15 minutes; carrying out inactivation reaction of reverse transcriptase under a condition of 85° C. for 5 seconds; and placing a product at 4° C.

Step III: Real-time quantitative PCR amplification detection, including following steps:

Step 1: preparing a real-time quantitative PCR reaction solution: including 25 uL of a PCR premix, 0˜10 ut of double distilled water (replenishing the system to 10 μwith water according to the total volume), 0˜2 ut of dye (for fluorescence compensation and correction of machine), 0.01 04 upstream primer of the CST1 gene, 0.01 04 downstream primer of the CST1 gene, 0.01 04 upstream primer of the reference gene, 0.01 04 downstream primer of the reference gene, 5 μof the cDNA, 1 μg of positive control, and 1 lig of negative control, wherein the positive control is a plasmid containing CST1, and the negative control is an empty plasmid (plasmid vector);

Step 2: adopting a two-step PCR amplification standard procedure, wherein the reaction condition of the two-step PCR amplification standard procedure includes the following steps: stage 1: pre-denaturation under a condition of 95° C. for 30 seconds; stage 2: PCR reaction under a condition of 95° C. for 15 seconds; and under a condition of 60° C. for 60 seconds, annealing and extension, 40 cycles in total as such.

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid; and

By comparing the difference between expression levels of the CST1 and the reference gene using the ΔCt method, it is obtained that a mean CT value of the CST1 is 21.5, and a mean CT value of the GAPDH is 18.0, then the ACT value is 3.5.

### EXAMPLE 9

Collecting and processing samples:

After a patient rinsed the nasal cavity with physiological saline, the surface of the nasal polyps were pressed by a brush (manufactured by Copan) under a nasal endoscope for 30 s, the brush was rotated for 3˜4 turns to brush the polyp surface, the brush was placed in a lysis solution, and stored at 4° C. for a short period (not exceeding 24 hours), or transferred to a temperature below -20° C. for long-term storage.

A method for detecting an expression level of a CST1 gene in nasal brushing cells, including following steps:

Step I: extracting RNA from nasal brushing cells;

Step 1: putting a genomic DNA adsorption column into a 2 mL collection tube, dissolving the nasal brushing cells in 2000 μL of a cell lysis solution, then adding the resultant into the genomic DNA adsorption column, centrifuging the resultant at 12000 r/min for 60 s, taking a filtrate, adding 70% ethanol in the same volume to the filtrate, mixing them uniformly, adding the resultant into an RNA purification column, centrifuging the resultant at 12000 r/min for 1 min, removing a filtrate, and placing the RNA purification column into a 2 mL collection tube;

Step 2: adding 700 μL of a first buffer solution to the RNA purification column obtained in step 1, centrifuging the resultant at 12000 r/min for 30 s, and removing a first filtrate; adding 800 μL of a second buffer solution to the RNA purification column continuously, centrifuging the resultant at 12000 r/min for 30 s, and removing a second filtrate;

Step 3: placing the RNA purification column from which the second filtrate was removed in step 2 in a 1.5 mL RNase-free collection pipe, adding 50 μL of RNase-free distilled water or 0.1% diethyl pyrocarbonate treated water to the RNA purification column, standing at room temperature for 5 minutes, centrifuging the resultant at 12000 r/min for 2 min, eluting the RNA purification column, and obtaining RNA, with measuring the ratio of OD260/OD280 of the RNA solution to be 2.0 using a spectrophotometer;

Step II: preparing cDNA by reverse transcription, including following steps: taking 3 μof the reverse transcription mixed solution, 0˜8 μL of the RNase-free and DNase-free water (replenishing the system to 8 μwith water according to the RNA amount), and total RNA in a total amount not exceeding 500 ng or a volume not exceeding 8 μ, wherein the RNase-free distilled water was used to replenish the system to 10 μL; gently mixing them uniformly and then carrying out reverse transcription reaction, under the following condition: carrying out the reverse transcription reaction under a condition of 37° C. for 15 minutes; carrying out inactivation reaction of reverse transcriptase under a condition of 85° C. for 5 seconds; and placing a product at 4° C.

Step III: Real-time quantitative PCR amplification detection, including following steps:

Step 1: preparing a real-time quantitative PCR reaction solution: including 5 μof a premix (containing enzyme required for PCR and a buffer solution), 0˜10 μL of double distilled water (replenishing the system to 10 μL with water according to the total volume), 0˜2 μL of dye (for fluorescence compensation and correction of machine), 1 μM upstream primer of the CST1 gene, 1 μM downstream primer of the CST1 gene, 1 μM upstream primer of the reference gene, 1 μM downstream primer of the reference gene, 2 μof the cDNA, 1 pg of positive control, and 1 μg of negative control;

Step 2: adopting a two-step PCR amplification standard procedure, wherein the reaction condition of the two-step PCR amplification standard procedure includes the following steps: stage 1: pre-denaturation under a condition of 95° C. for 30 seconds; stage 2: PCR reaction under a condition of 95° C. for 15 seconds, and under a condition of 60° C. for 60 seconds, annealing and extension, 40 cycles in total as such.

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid; and

By comparing the difference between expression levels of the CST1 and the reference gene using the ΔCt method, it is obtained that a mean CT value of the CST1 is 25.1, and a mean CT value of GAPDH is 17.9, then the ACT value is 7.2.

In the above Examples 7˜9 of the present disclosure, preferably, the first buffer solution used, RWA buffer, is manufactured by Takara, Article No. 9767; and the second buffer solution, RWB buffer, is manufactured by Takara, Article No. 9767. However, the scope of protection of the present disclosure is not limited to the above first buffer solution and second buffer solution. A person skilled in the art might make a selection according to actual application requirements.

### EXAMPLE 10

Collecting and processing samples:

78 subjects rinsed the nasal cavities with physiological saline, then the surface of the nasal polyps were pressed by a brush (manufactured by Copan) under a nasal endoscope for 30 s, the brush was rotated for 3˜4 turns to brush the polyp surface, the brush was placed in a subsequent lysis solution, and stored at 4° C. for a short period (not exceeding 24 hours), or transferred to a temperature below -20° C. for long-term storage.

A method for detecting an expression level of a CST1 gene in nasal brushing cells, including following steps:

Step I: extracting RNA from nasal brushing cells; to a centrifuge tube with nasal mucosa exfoliated cells adding 1 mL of an RNA extraction solution to dissolve and shake, then standing at room temperature for 5 min; adding 200 μL of chloroform, shaking and mixing the resultant uniformly, standing at room temperature for 5 min, and centrifuging the resultant at 12000 r/min at 4° C. for 15 min; obtaining 200 μL of a supernatant, adding 200 μL of isopropyl, mixing them uniformly and then standing for 10 min, centrifuging the resultant at 12000 r/min at 4° C. for 15 min, discarding the supernatant, and retaining a first precipitate; to the first precipitate adding 75% ethanol in a volume equal to that of isopropanol, centrifuging the resultant at 7500 r/min at 4° C. for 15 minutes, discarding a supernatant, and retaining a second precipitate; capping the centrifuge tube tightly, and centrifuging the resultant at 7500 r/min at 4° C. for 2 min, removing the supernatant, standing for 15 min, then continuously adding 50 μL of RNase-free and DNase-free water to the centrifuge tube to dissolve the second precipitate, and obtaining RNA, wherein a ratio of OD260/OD280 of the RNA solution was measured to be 1.8 using a spectrophotometer;

Step II: preparing cDNA by reverse transcription, through the same steps as those in Example 7;

Step III: real-time quantitative PCR amplification detection, through the same steps as those in Example 7;

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid;

Step 1: calculating mean ACT of the healthy control group:

In the present example, subjects 1˜10 were in the healthy control group, and a calculation method is as follows: mean ACT (CT(CST1)-CT(GAPDH))=(2.999+3.401+6.268+4.141+4.173+2.832+3.225+2.135+3.512)/10=3.632;

Step 2: calculating a relative expression level of the subjects:

Taking 3.632 as a reference, the relative expression level of each subject was obtained (indicating expression level of the present gene relative to the mean value of the healthy control group in the subject, wherein the numerical value represents the times of change, for example, 1.5 means that the expression level of the subject is 1.5 times the mean value of the healthy control group). The calculation results are shown in Table 3.

A calculation formula is 2.^{-ΔΔCt}, −ΔΔCT=−(ΔCT (subject CT(CST1)-CT(GAPDH))-mean ΔCT (mean ΔCT of healthy control group)).

TABLE 3
Calculation Results of CSTI Gene Expression
Level of the Method Provided in Example 8

| | | | | | Relative |
|---|---|---|---|---|---|
| Subject | CST1 CT | GAPDH CT | | | Expression |
| No. | Value | Value | ΔCT | −ΔΔCT | Level |
| 1 | 20.516 | 17.517 | 2.999 | 0.633 | 1.550786 |
| 2 | 22.083 | 18.682 | 3.401 | 0.231 | 1.173648 |
| 3 | 24.481 | 18.213 | 6.268 | −2.636 | 0.160874 |
| 4 | 22.426 | 18.285 | 4.141 | −0.509 | 0.702709 |
| 5 | 25.918 | 21.746 | 4.173 | −0.541 | 0.687294 |
| 6 | 23.726 | 20.894 | 2.832 | 0.8 | 1.741101 |
| 7 | 21.844 | 18.619 | 3.225 | 0.407 | 1.325926 |
| 8 | 23.134 | 20.999 | 2.135 | 1.497 | 2.822552 |
| 9 | 18.848 | 15.336 | 3.512 | 0.12 | 1.086735 |
| 10 | 20.408 | 17.45 | 2.958 | 0.674 | 1.59549 |
| 11 | 17.97 | 18.401 | −0.431 | 4.063 | 16.71417 |
| 12 | 23.999 | 23.591 | 0.408 | 3.224 | 9.343739 |
| 13 | 16.721 | 18.432 | −1.711 | 5.343 | 40.58852 |
| 14 | 22.541 | 21.136 | 1.405 | 2.227 | 4.681595 |
| 15 | 16.253 | 17.14 | −0.887 | 4.519 | 22.92739 |
| 16 | 17.001 | 17.213 | −0.212 | 3.844 | 14.36016 |
| 17 | 17.724 | 17.679 | 0.045 | 3.587 | 12.01696 |
| 18 | 28.194 | 25.744 | 2.45 | 1.182 | 2.268911 |
| 19 | 14.361 | 15.182 | −0.821 | 4.453 | 21.90214 |
| 20 | 13.925 | 14.416 | −0.491 | 4.123 | 17.42395 |
| 21 | 24.851 | 24.904 | −0.053 | 3.685 | 12.86162 |
| 22 | 28.412 | 24.025 | 4.388 | −0.756 | 0.592136 |
| 23 | 18.363 | 17.402 | 0.961 | 2.671 | 6.368705 |
| 24 | 24.828 | 18.832 | 5.996 | −2.364 | 0.194252 |
| 25 | 16.164 | 16.671 | −0.507 | 4.139 | 17.61827 |
| 26 | 19.158 | 19.809 | −0.651 | 4.283 | 19.46756 |
| 27 | 24.782 | 15.703 | 9.079 | −5.447 | 0.022924 |
| 28 | 25.289 | 18.756 | 6.533 | −2.901 | 0.133879 |
| 29 | 18.573 | 18.253 | 0.32 | 3.312 | 9.93142 |
| 30 | 13.275 | 14.892 | −1.617 | 5.249 | 38.02826 |
| 31 | 28.409 | 19.394 | 9.015 | −5.383 | 0.023964 |
| 32 | 16.912 | 20.249 | −3.337 | 6.969 | 125.2789 |
| 33 | 27.906 | 23.371 | 4.535 | −0.903 | 0.534774 |
| 34 | 24.602 | 21.954 | 2.649 | 0.983 | 1.976571 |
| 35 | 27.754 | 24.971 | 2.783 | 0.849 | 1.801252 |
| 36 | 17.838 | 16.58 | 1.258 | 2.374 | 5.183764 |
| 37 | 22.207 | 18.892 | 3.315 | 0.317 | 1.245737 |
| 38 | 18.172 | 16.958 | 1.214 | 2.418 | 5.344296 |
| 39 | 23.533 | 15.102 | 8.431 | −4.799 | 0.035922 |
| 40 | 17.576 | 24.94 | −7.364 | 10.996 | 2042.33 |
| 41 | 18.868 | 19.801 | −0.933 | 4.565 | 23.6702 |
| 42 | 20.53 | 17.544 | 2.986 | 0.646 | 1.564824 |
| 43 | 19.043 | 20.585 | −1.542 | 5.174 | 36.10183 |
| 44 | 23.517 | 22.84 | 0.677 | 2.955 | 7.754319 |
| 45 | 21.723 | 17.992 | 3.731 | −0.099 | 0.93368 |
| 46 | 26.237 | 24.259 | 1.978 | 1.654 | 3.14705 |
| 47 | 20.548 | 18.932 | 1.616 | 2.016 | 4.044608 |
| 48 | 19.051 | 15.53 | 3.521 | 0.111 | 1.079977 |
| 49 | 25.902 | 17.331 | 8.571 | −4.939 | 0.0326 |
| 50 | 25.413 | 18.73 | 6.683 | −3.051 | 0.120658 |
| 51 | 19.333 | 17.993 | 1.34 | 2.292 | 4.897346 |
| 52 | 23.645 | 18.306 | 5.339 | −1.707 | 0.306296 |
| 53 | 27.97 | 19.675 | 8.296 | −4.664 | 0.039445 |
| 54 | 16.468 | 19.083 | −2.615 | 6.247 | 75.95116 |
| 55 | 22.416 | 19.957 | 2.46 | 1.172 | 2.253238 |
| 56 | 19.835 | 19.533 | 0.302 | 3.33 | 10.05611 |
| 57 | 20.906 | 20.823 | 0.083 | 3.549 | 11.70457 |
| 58 | 22.729 | 19.604 | 3.125 | 0.507 | 1.421092 |
| 59 | 16.791 | 18.483 | −1.692 | 5.324 | 40.05749 |
| 60 | 13.83 | 17.371 | −3.541 | 7.173 | 144.3073 |
| 61 | 17.152 | 18.704 | −1.552 | 5.184 | 36.35294 |
| 62 | 16.108 | 17.418 | −1.31 | 4.942 | 30.73904 |
| 63 | 17.612 | 16.936 | 0.676 | 2.956 | 7.759695 |
| 64 | 16.67 | 17.256 | −0.586 | 4.218 | 18.60992 |
| 65 | 25.193 | 20.368 | 4.825 | −1.193 | 0.437392 |
| 66 | 29.616 | 26.508 | 3.108 | 0.524 | 1.437937 |
| 67 | 16.03 | 17.129 | −1.099 | 4.731 | 26.55663 |
| 68 | 20.07 | 20.927 | −0.857 | 4.489 | 22.45555 |
| 69 | 24.713 | 22.608 | 2.105 | 1.527 | 2.881859 |
| 70 | 25.641 | 19.255 | 6.386 | −2.754 | 0.148239 |
| 71 | 25.259 | 17.487 | 7.772 | −4.14 | 0.05672 |
| 72 | 26.232 | 15.977 | 10.255 | −6.623 | 0.010146 |
| 73 | 27.171 | 18.604 | 8.567 | −4.935 | 0.03269 |
| 74 | 22.065 | 21.104 | 0.961 | 2.671 | 6.368705 |
| 75 | 23.583 | 16.853 | 6.73 | −3.098 | 0.116791 |
| 76 | 23.957 | 15.071 | 8.886 | −5.254 | 0.026205 |
| 77 | 26.333 | 20.924 | 5.409 | −1.777 | 0.29179 |
| 78 | 19.705 | 17.984 | 1.721 | 1.911 | 3.760697 |

### EXAMPLE 11

Collecting and processing samples:

Patients were ordered to rinse the nasal cavities with physiological saline before acquiring cells, the surface of the nasal polyps were pressed by a brush (manufactured by Copan) under a nasal endoscope for 30 s, the brush was rotated for 3˜4 turns to brush the polyp surface, the brush was placed in a lysis solution, and stored at 4° C. for a short period (not exceeding 24 hours), or transferred to a temperature below −20° C. for long-term storage.

A method for detecting an expression level of a CST1 gene in nasal brushing cells, including following steps:

Step I: a step of extracting RNA from nasal brushing cells: to a centrifuge tube with nasal brushing cells adding 20 mL of an RNA extraction solution to dissolve and shake, then standing at room temperature for 7 min; adding 10 mL of chloroform, shaking and mixing the resultant uniformly, standing at room temperature for 7 min, and centrifuging the resultant at 14000 r/min at 5° C. for 20 min; taking 20 mL of a supernatant, adding 20 mL of isopropyl, mixing them uniformly and then standing for 12 min, centrifuging the resultant at 14000 r/min at 5° C. for 20 min, discarding the supernatant, and retaining a first precipitate; to the first precipitate adding 40 mL of 90% ethanol, centrifuging the resultant at 14000 r/min, at 5° C., for 3 minutes, discarding a supernatant, and retaining a second precipitate; capping the centrifuge tube tightly, and centrifuging the resultant at 14000 r/min at 5° C. for 3 min, removing the supernatant, standing for 20 min, then continuously adding 5 mL of RNase-free and DNase-free water to the centrifuge tube to dissolve the second precipitate, and obtaining RNA, wherein a ratio of OD260/OD280 of the RNA solution was measured to be 2.1 using a spectrophotometer;

Step II: preparing cDNA by reverse transcription, through the same steps as those in Example 7;

Step III: real-time quantitative PCR amplification detection, in which step a real-time quantitative PCR reaction solution was prepared in the same manner as in Example 7, and a three-step method was adopted for the PCR amplification standard procedure, wherein reaction condition of the three-step PCR amplification standard procedure included the following steps: stage 1: pre-denaturation under a condition of 95° C. for 2 minutes; stage 2: PCR reaction under a condition of 95° C. for 1 minute, under a condition of 55° C. for 1 minute, and under a condition of 72° C. for 1 minute, 40 cycles as such; and finally, annealing and extension at 72° C. for 7 minutes.

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid; and

Mean Ct of the positive control wells is 16.2, mean Ct of the negative control wells is 39.7, mean Ct of the sample CST1 is 17.8, and mean Ct of the sample GAPDH is 16.4, then the difference value is 17.8˜16.4, namely 1.4, indicating that this patient's CST1 expression is 0.51 times that of the GAPDH (1/2¹⁴).

### EXAMPLE 12

Collecting and processing samples:

Patients were ordered to rinse the nasal cavities with physiological saline before acquiring cells, the surface of the nasal polyps were pressed by a brush (manufactured by Copan) under a nasal endoscope for 30 s, the brush was rotated for 3˜4 turns to brush the polyp surface, the brush was placed in a lysis solution, and stored at 4° C. for a short period (not exceeding 24 hours), or transferred to a temperature below -20° C. for long-term storage.

A method for detecting an expression level of a CST1 gene in nasal brushing cells, including following steps:

Step I: a step of extracting RNA from nasal brushing cells: to a centrifuge tube with nasal brushing cells adding 0.1 mL of an RNA extraction solution to dissolve and shake, then standing at room temperature for 7 min; adding 0.03 mL of chloroform, shaking and mixing the resultant uniformly, standing at room temperature for 7 min, and centrifuging the resultant at 14000 r/min at 5° C. for 20 min; taking 20 mL of a supernatant, adding 0.015 mL of isopropyl, mixing them uniformly and then standing for 12 min, centrifuging the resultant at 14000 r/min at 5° C. for 20 min, discarding the supernatant, and retaining a first precipitate; to the first precipitate adding 0.0075 mL of 90% ethanol, centrifuging the resultant at 14000 r/min at 5° C. for 3 minutes, discarding a supernatant, and retaining a second precipitate; capping the centrifuge tube tightly, and centrifuging the resultant at 14000 r/min at 5° C. for 3 min, removing the supernatant, standing for 20 min, then continuously adding 0.01 mL of RNase-free and DNase-free water to the centrifuge tube to dissolve the second precipitate, and obtaining RNA, wherein a ratio of OD260/OD280 of the RNA solution was measured to be 2.1 using a spectrophotometer;

Step II: preparing cDNA by reverse transcription, through the same steps as those in Example 7;

Step III: real-time quantitative PCR amplification detection, in which step a real-time quantitative PCR reaction solution was prepared in the same manner as in Example 7, and a three-step method was adopted for the PCR amplification standard procedure, wherein reaction condition of the three-step PCR amplification standard procedure included the following steps: stage 1: pre-denaturation under a condition of 95° C. for 2 minutes; stage 2: PCR reaction under a condition of 95° C. for 1 minute, under a condition of 55° C. for 1 minute, and under a condition of 72° C. for 1 minute, 40 cycles as such; and finally, annealing and extension at 72° C. for 7 minutes.

Step IV: calculating the expression level of the CST1 gene:

After the reaction was completed, an amplification curve and a melting curve of the real-time quantitative PCR were confirmed, the Ct values of the CST1 and the GAPDH were read, analysis was performed using the ΔCt analysis method (the Ct value of the CST1 minus the Ct value of the GAPDH), and the GAPDH was used as the reference gene; the experiment was considered valid if a positive control Ct value is less than 20 and a negative control Ct value is greater than 38, otherwise the experiment was invalid; and

Mean Ct of the positive control wells is 15.9, mean Ct of the negative control wells is 38.7, mean Ct of the sample CST1 is 18.8, and mean Ct of the sample GAPDH is 16.4, then the difference value is 18.8˜16.4, namely 2.4, indicating that this patient's CST1 expression is 0.51 times that of the GAPDH (1/2²⁴).

### EXAMPLE 13

I. Research Method

1. Enrolling Patients and Hormone Sensitivity Evaluation Index:

Enrolling patients with chronic rhinosinusitis with nasal polyps: clinical information (sex, age, concomitant diseases, symptom score, sinuses CT Lund-Mackay score, polyp size score before hormone treatment, etc.) of patients with chronic rhinosinusitis complying with EPOS 2012 diagnosis standard and without contraindications to oral glucocorticoid was collected.

Each patient was subjected to oral hormone treatment (methylprednisolone 24 mg, taken at a draught in the morning) for 2 weeks, and the polyp sizes were scored again after the oral hormone treatment was ended. The patients were classified into a glucocorticoid sensitive group (the polyp size score is decreased by no less than 1 point) and a glucocorticoid insensitive group (the polyp size score is not decreased by more than 1 point) according to the changes in polyp scores of patients before and after the oral hormone treatment. The scoring standard is as follows:

TABLE 4
Table of Nasal Polyp Size Scores

| Polyp Score | Size Description |
|---|---|
| 0 | no polyp |
| 1 | small polyp confined in middle nasal meatus and not |
| | beyond inferior border of middle turbinate |
| 2 | polyp reaching inferior border of middle turbinate |
| 3 | large polyp beyond upper border of inferior turbinate |
| 4 | large polyp almost completely blocking nasal cavity |

Note: the scores of all patients enrolled on a single side were not less than 2 points before oral hormone treatment.

2. Collecting Specimens:

Collecting nasal secretions: collecting the patients' nasal cavity secretions by an expansion sponge absorption method (gently placing a piece of expansion sponge, in a size of 0.5cm×3cm, between inferior turbinate and septum of the patients, with an upper end of the expansion sponge facing to a margo liber of the middle turbinate, 5 min later, taking out the sponge for subsequent use) before starting the oral hormone treatment.

Collecting nasal brushing cells: sampling at the middle nasal meatus under the guidance of a nasal endoscope: gently pressing the surface of mucosa in the middle nasal meatus with a sterile cotton swab for 30 seconds, and rotating the cotton swab for 5 turns under the nasal endoscope, then placing the swab in a sterile collection tube containing a transport medium, breaking it immediately at a red line, and marking information and storing the swab at −80° C.

Collecting polyp tissue specimens: taking small pieces of nasal polyp tissues with sterile ethmoid forceps for subsequent use.

3. Specimen Processing and Experimental Method:

Nasal secretions: placing the expansion sponge absorbed with the nasal secretions into a 15 mL centrifuge tube, adding 500 μL of physiological saline, standing at 4° C. for 2 h, then placing the expansion sponge into a syringe with needle and piston removed, placing the syringe into the original centrifuge tube to centrifuge at 1500 rpm at 4° C. for 15 min, and taking a supernatant as the patient's nasal secretion specimen. A total protein content of the nasal secretion specimen was measured by a BCA method and the specimen was diluted to a total protein content of 5×10⁻³ mg/mL, and a CST1 content thereof was measured by an ELISA method (Cloud-Clone Corp. USA, with a detection range of 0.2 ng/mL˜10 ng/mL).

Nasal brushing cells: extracting the total RNA by a conventional method, making RNA to undergo reverse transcription to cDNA, and performing fluorescence quantitative PCR to detect the expression of the CST1 and the reference gene (such as GAPDH).

Extracting total RNA: hereinafter, an extraction method is described by taking a Trizol reagent as an example, and any method capable of extracting the total RNA may be applicable. (1) Adding 1 mL of Trizol to a test tube with exfoliated cells, shaking the test tube, then adding 200 μof chloroform (trichloromethane), and vigorously shaking and mixing them uniformly, and standing at room temperature for 5 minutes; (2) centrifuging the resultant at 12000 rpm at 4° C. for 15 min; (3) adding about 250 μL of a supernatant to an EP tube, adding an equal volume (about 250 μL) of isopropanol, mixing them uniformly and then standing for 10 minutes, centrifuging the resultant at 12000 rpm at 4° C. for 15 min, discarding the supernatant, and retaining the precipitate, (4) adding 75% ethanol in a volume (about 250 μL) equal to that of isopropanol to clean the precipitate, mixing them uniformly, centrifuging the resultant at 7500 rpm at 4° C. for 15 min, discarding the supernatant, and retaining the precipitate, (5) opening the cover, and placing the resultant in a fuming cupboard for 15 minutes to volatilize the ethanol; and (6) adding 20˜100 μL of RNase-free water to dissolve the precipitate, and measuring RNA concentration using a spectrophotometer.

Making RNA to undergo reverse transcription to cDNA: similarly, only an example is given herein, while any reagent capable of making the total RNA to undergo reverse transcription to cDNA may be used for this reaction. A Takara's reverse transcription kit (Article No. RR036A) was taken as an example, and a reaction system included 2 μof prime mix (a reagent was included in the kit), and 500 ng of total RNA, and was replenished to 10 μL with RNase Free dH2O. The cDNA was obtained after reacting at 37° C. for 15 min.

Detecting the expression of the CST1 and the reference gene GAPDH by fluorescence quantitative PCR: similarly, any primer capable of detecting the expression level of the CST1 or the expression level of the reference gene GAPDH is applicable to the present method. In addition, any reagent capable of performing fluorescence quantitative PCR is applicable to the present method. A Takara's fluorescence quantitative PCR kit (Article No. RR820A) is taken as an example below.

TABLE 5
Target Gene Primers

| | | |
|---|---|---|
| CST1-forward | SEQ ID | 5′-TAGGATAATCCCGGGTGGCA-3′ |
| | NO. 2 | |
| CST1-reverse | SEQ ID | 5′-GTCTGTTGCCTGGCTCTTAGT-3′ |
| | NO. 3 | |
| GAPDH-forward | SEQ ID | 5′-CTCCTCCTGTTCGACAGTCAGC-3′ |
| | NO. 4 | |
| GAPDH-reverse | SEQ ID | 5′-CCCAATACGACCAAATCCGTT-3′ |
| | NO. 5 | |

TABLE 6
Reaction System

| Reactant | Dosage |
|---|---|
| SYBR Green 1 | 5 μL |
| forward primer (10 μM) | 0.5 μL |
| reverse primer (10 μM) | 0.5 μL |
| ROX | 0.2 μL |
| cDNA or positive control or | 1 ng, |
| negative control | 1 μL in volume |
| ddH2O | 2.8 μL |

Reaction condition for performing the PCR amplification (a three-step method or a two-step method may be chosen).

Three-step method: pre-denaturation at 95° C. for 2 minutes, followed by 40 cycles of PCR reaction at 95° C. for 1 minute, 55° C. for 1 minute, and 72° C. for 1 minute, and finally extension at 72° C. for 7 minutes.

Two-step method: pre-denaturation at 95° C. for 30 seconds, PCR reaction at 95° C. for 5 seconds, and annealing and extension at 60° C. for 1 minute, 40 cycles in total.

Experimental result interpretation: the Ct value of the target gene CST1 minus the Ct value of the reference gene (such as GAPDH) is defined as ΔCt, wherein the lower the ΔCt value is, the higher the expression level of the CST1 in the exfoliated cells is.

Nasal polyp tissues: after being fixed by a tissue fixing solution, the tissues were embedded with paraffin, cut into 5 μm sections by Leica RM2235 cryostat (Leica Microsystems, Bannockburn, Ill., USA) slicer and then underwent HE staining, percentages of inflammatory cells (eosinophils, neutrophils, lymphocytes and plasma cells) were counted under a 400-fold microscope visual field, 3 non-overlapping visual fields were counted for each section, 5 sections were counted for each polyp tissue case, and a mean value of the 15 visual fields was taken as the inflammatory cell infiltration condition of this polyp tissue case.

II. Predicting Hormone Sensitivity for Chronic Rhinosinusitis with Nasal Polyps According to CST1 Content in Nasal Secretions

Enrolling patients: 111 patients with chronic rhinosinusitis with nasal polyps complying with EPOS 2012 diagnosis standard and without contraindications to oral glucocorticoid were enrolled in total, clinical information (sex, age, concomitant diseases, symptom score, sinuses CT Lund-Mackay score, polyp size score before hormone treatment, etc.) was collected according to the preceding method, and oral hormone treatment (methylprednisolone 24 mg, taken at a draught in the morning) was carried out for 2 weeks.

Nasal secretions: acquisition, specimen processing and detection methods are the same as the above.

Experimental Results:

1. Clinical information and laboratory data for the glucocorticoid sensitive group and the glucocorticoid insensitive group indicate that the glucocorticoid sensitive group and the glucocorticoid insensitive group have differences in indexes such as CST1 concentration, percentage of eosinophils of nasal polyp tissues, percentage of neutrophils of nasal polyp tissues, percentage of lymphocytes of nasal polyp tissues, percentage of plasma cells of nasal polyp tissues, and CT ethmoidal sinus score/maxillary sinus score before treatment, in the patients' nasal secretions. (Table 7)

TABLE 7
Patients' Clinical Information and Laboratory Results

| | Glucocorticoid | Glucocorticoid | |
|---|---|---|---|
| | sensitive Group | insensitive Group | |
| | (75 cases) | (36 cases) | p Value |
| sex (male/female) | 43/32 | 27/9 | 0.0932 |
| age (mean value ± | 43.27 ± 12.57 | 46.92 ± 11.78 | 0.2911 |
| standard deviation) | | | |
| nasal obstruction (median; | 7.0; 6.0-8.0 | 7.0; 6.0-8.0 | 0.6970 |
| quartile) | | | |
| rhinorrhea (median; quartile) | 5.0; 4.0-6.0 | 5.0; 4.0-6.75 | 0.9077 |
| headache and facial | 0; 0-4.0 | 0; 0-2.75 | 0.5979 |
| pain (median; quartile) | | | |
| CT score before treatment | 19.0; 14.0-21.0 | 16.5; 14.0-20.0 | 0.2547 |
| (median; quartile) | | | |
| CT ethmoidal sinus score/ | 2.3; 2.0-3.5 | 2.0; 2.0-2.5 | 0.0048* |
| maxillary sinus score before | | | |
| treatment (median; quartile) | | | |
| nasal endoscope score | 5.0; 4.0-7.0 | 6.0; 4.25-6.0 | 0.8451 |
| (before hormone treatment) | | | |
| (median; quartile) | | | |
| percentage % of tissue | 57.99; 37.74-70.30 | 5.0; 2.35-26.90 | <0.0001* |
| eosinophils (median; quartile) | | | |
| percentage % of tissue | 0; 0-1.30 | 2.35; 0.125-8.675 | <0.0001* |
| neutrophils (median; quartile) | | | |
| percentage % of tissue | 23.00; 13.70-38.10 | 42.55; 30.49-60.56 | <0.0001* |
| lymphocytes (median; quartile) | | | |
| percentage % of tissue plasma | 15.00; 7.00-23.00 | 25.45; 11.75-44.95 | 0.0003* |
| cells (median; quartile) | | | |
| percentage % of peripheral | 6.30; 4.80-8.40 | 3.55; 1.65-6.30 | <0.0001* |
| blood eosinophils (median; | | | |
| quartile) | | | |
| percentage % of peripheral | 53.00; 47.00-58.10 | 57.55; 48.93-62.08 | 0.0566 |
| blood neutrophils (median; | | | |
| quartile) | | | |
| CST1 concentration (ng/mL) | 5220; 3260-7480 | 228.5; 0.2-902.8 | <0.0001* |
| in nasal secretions (median; | | | |
| quartile) | | | |

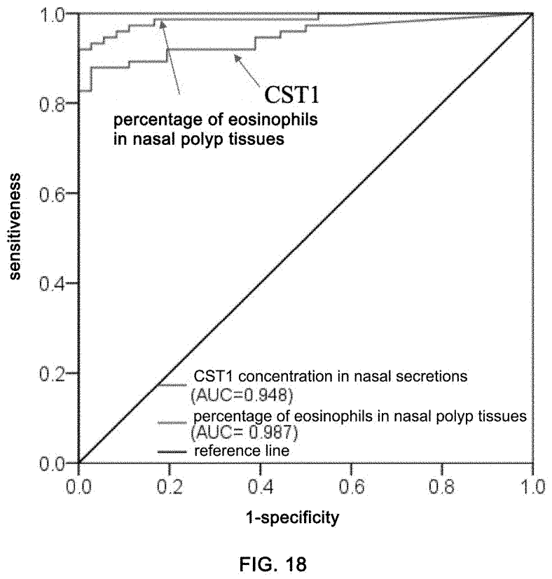

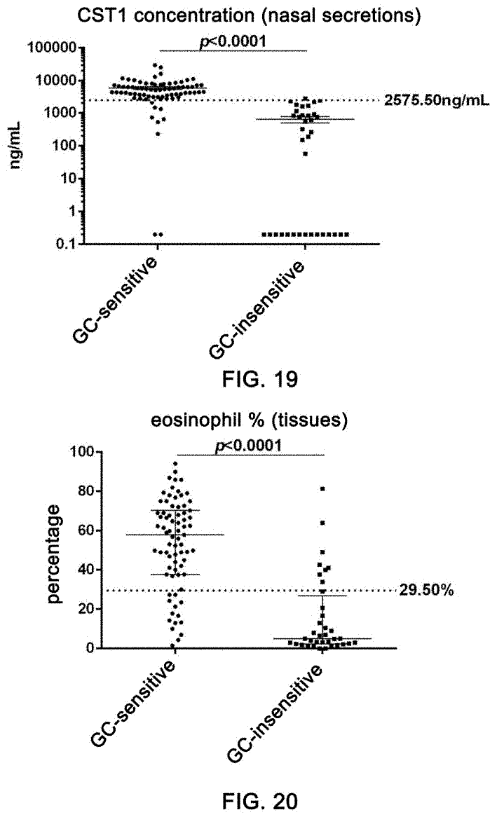

2. Incorporating all of the difference variables in Result 1 into logistic regression analysis shows that only the CST1 concentration in the nasal secretions and the percentage of eosinophils in polyp tissues can predict sensitivity of oral hormone treatment. Therefore, a receiver operating characteristic curve (ROC curve) (FIG. 18) was made, which indicates that the prediction accuracy of the CST1 concentration in the nasal secretions is 94.8%, and the prediction accuracy of the tissue eosinophils is 98.7%, thus the two have comparable prediction efficacies (statistical detection of P >0.05). (FIG. 19) The sensitiveness of the CST1 concentration in the nasal secretions to predict the sensitivity of oral hormone treatment is 0.880, the specificity is 0.972, and the prediction threshold value is 2575.5 ng/mL; (FIG. 20) the sensitiveness of the percentage of eosinophils in the polyp tissues to predict the sensitivity of oral hormone treatment is 0.920, the specificity is 1, and the prediction threshold value is 29.5%.

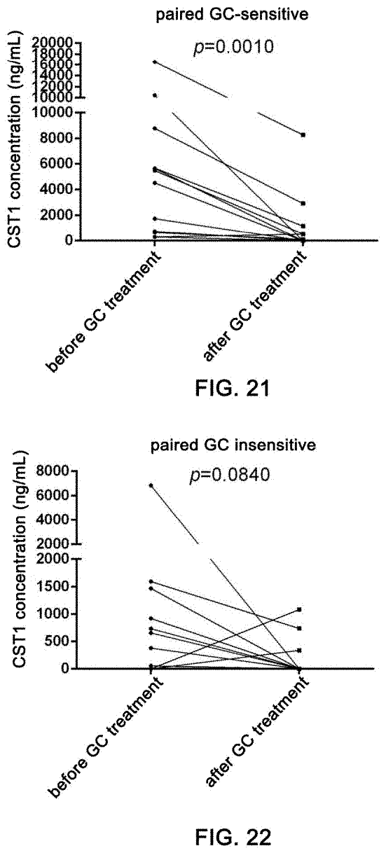

3. The oral hormone treatment may reduce the CST1 concentration in the nasal secretions of the hormone-sensitive patients (FIG. 21), but the oral hormone treatment has no statistical difference in the effect on CST1 concentration in the nasal secretions of the hormone-insensitive patients (FIG. 22).

III. Predicting Hormone Sensitivity for Chronic Rhinosinusitis with CST1 Content in Nasal brushing cells

Enrolling patients: 83 patients with chronic rhinosinusitis with nasal polyps complying with EPOS 2012 diagnosis standard and without contraindications to oral glucocorticoid were enrolled in total, clinical information (sex, age, concomitant diseases, symptom score, sinuses CT Lund-Mackay score, polyp size score before hormone treatment, etc.) was collected according to the preceding method, and oral hormone treatment (methylprednisolone 24 mg, taken at a draught in the morning) was carried out for 2 weeks.

Nasal brushing cells: acquisition, specimen processing and detection methods are the same as the above.

Experimental Results:

1. Clinical information and laboratory data for the glucocorticoid sensitive group and the glucocorticoid insensitive group indicate that the glucocorticoid sensitive group and the glucocorticoid insensitive group have differences in indexes such as CST1 value (ΔCt), percentage of eosinophils of nasal polyp tissues, percentage of neutrophils of nasal polyp tissues, percentage of lymphocytes of nasal polyp tissues, percentage of plasma cells of nasal polyp tissues, and CT ethmoidal sinus score/maxillary sinus score before treatment, in the patients' nasal brushing cells. (Table 8)

TABLE 8
Patients' Clinical Information and Laboratory Results

| | Glucocorticoid | Glucocorticoid | |
|---|---|---|---|
| | sensitive Group | insensitive Group | |
| | (49 cases) | (34 cases) | p Value |
| sex (male/female) | 29/20 | 22/12 | 0.258 |
| age (mean value ± | 43.5 ± 12.4 | 47.8 ± 13.4 | 0.164 |
| standard deviation) | | | |
| nasal obstruction | 7.0; 5.0-8.0 | 7.0; 4.0-8.0 | 0.101 |
| (median; quartile) | | | |
| rhinorrhea (median; | 5.0; 4.0-6.0 | 4.0; 2.0-6.0 | 0.200 |
| quartile) | | | |
| headache and facial | 1.0; 0-4.0 | 0; 0-3 | 0.264 |
| pain (median; | | | |
| quartile) | | | |
| CT score before | 20.0; 14.0-23.0 | 18; 12.0-21.0 | 0.250 |
| treatment (median; | | | |
| quartile) | | | |
| CT ethmoidal sinus | 2.0; 2.0-3.0 | 2.0; 1.5-2.3 | 0.035* |
| score/maxillary | | | |
| sinus score before | | | |
| treatment (median; | | | |
| quartile) | | | |
| nasal endoscope | 5.0; 4.0-7.0 | 6.0; 4.0-6.0 | 0.8125 |
| score (before | | | |
| hormone treatment) | | | |
| (median; quartile) | | | |
| percentage % of | 65.1; 41.4-77.2 | 3.8; 1.2-11.8 | <0.0001* |
| tissue eosinophils | | | |
| (median; quartile) | | | |
| percentage % of | 0; 0-1.2 | 2.3; 0.0-26.1 | <0.0001* |
| tissue neutrophils | | | |
| (median; quartile) | | | |
| percentage % of | 19.6; 9.2-28.1 | 48.3; 27.8-74.4 | <0.0001* |
| tissue lymphocytes | | | |
| (median; quartile) | | | |
| percentage % of | 10.7; 7.1-22.1 | 21.3; 9.4-33.0 | 0.004* |
| tissue plasma cells | | | |
| (median; quartile) | | | |
| percentage % of | 3.30; 6.5-9.5 | 2.7; 1.3-3.6 | <0.0001* |
| peripheral blood | | | |
| eosinophils (median; | | | |
| quartile) | | | |
| percentage % of | 53.00; 46.3-57.50 | 58.0; 55.4-61.4 | 0.004 |
| peripheral blood | | | |
| neutrophils (median; | | | |
| quartile) | | | |
| CST1 value (ΔCt) in | 0.2; −1.0-2.5 | 7.1; 2.2-8.1 | <0.0001* |
| nasal brushing cells | | | |
| (median; quartile) | | | |

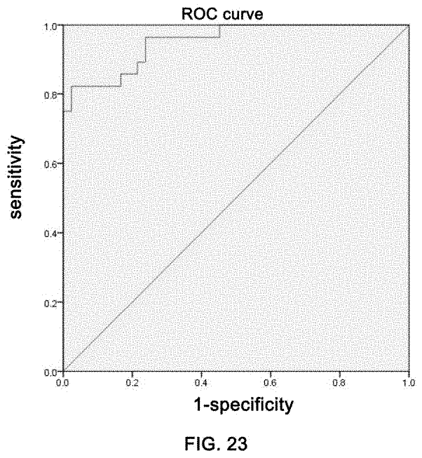

2. Incorporating all of the difference variables in 1 into logistic regression analysis shows that only the CST1 value (ΔCt) in the nasal brushing cells and the percentage of eosinophils in the polyp tissues can predict the sensitivity of oral hormone treatment. Therefore, a receiver operating characteristic curve (ROC curve) (FIG. 23) was made, and an area below the ROC curve indicates that the prediction accuracy of the CST1 value (ΔCt) in the nasal brushing cells is 95.2%.

Area below the curve

test result variable: VAR00001

| | | | Progressive 95% | |
|---|---|---|---|---|
| | Standard | Progressive | Confidence Interval | |
| Area | Error ^{a} | Significance ^{b} | Lower Limit | Upper Limit |
| .952 | .024 | .000 | .905 | .998 |

on nonparametric assumption

original hypothesis : true area =0.5

The above experiments show:

The CST1 concentration in the nasal secretions and the nasal brushing cells can predict CRSwNP patients' sensitivity to oral hormone treatment. The sample collection method thereof is safe and non-invasive, and has good tolerability, and the detection method is simple and fast, and has good prediction performance, thus having a good clinical application prospect.

Although the present disclosure has been illustrated and described with specific examples, it should be appreciated that many other changes and modifications may be made without departing from the spirit and scope of the present disclosure. Thus, this means that all such changes and modifications that belong to the scope of the present disclosure are included in the appended claims.

### INDUSTRIAL APPLICABILITY

1. The present disclosure provides a kit for detecting chronic rhinosinusitis with nasal polyps subtype. Through screening by proteomics and transcriptomics methods, the CST1 gene is adopted as a biomarker, and applied to the kit, so as to realize the method for detecting chronic rhinosinusitis with nasal polyps subtype with the kit, so that the finally obtained kit includes the specific primer of the CST1 gene. On the basis of having this specific primer, the kit of the present disclosure can rapidly identify nasal polyp subtype, and has higher accuracy compared with the conventional pathological detection method. This kit may perform mass and rapid detection simultaneously on the samples, thus saving the labor costs and medical costs. Moreover, the systematic kit has higher identification accuracy, may comprehensively reflect histopathological characteristics, thus solving the influence of human error in the prior art, and avoiding the drawback of misdiagnosis caused by the fact that the tissue section reflects local characteristics of the tissues. Rapidly, accurately, and comprehensively identifying the nasal polyp subtype with the kit is of vital importance to clinical diagnosis and treatment, so as to perform targeted treatment according to the inflammation subtype of nasal polyps as soon as possible, effectively guide the determination of the medication mode and operation mode for the patients with chronic rhinosinusitis with nasal polyps, accurately estimate the response to medication, and judge the prognosis effect.

2. The kit provided in the present disclosure can obtain the nasal polyp cells from the surface of nasal polyps in a manner of brushing or sticking for detection, so as to determine the chronic rhinosinusitis with nasal polyps subtype of the patient, avoid wounds to the patient, and improve the safety of patient examination, moreover, the operation is more convenient, and labor costs and medical costs are saved.

Compared with the prior art, the advantages and positive effects of the present disclosure lie in:

3. the method for detecting an expression level of a CST1 gene in nasal brushing cells provided in the present disclosure, taking the effectively screened CST1 gene as a biomarker, provides a method for detecting the expression level of the gene, realizes the calculation of the expression level of the CST1 gene in the nasal brushing cells, and can effectively acquire the expression level of the CST1 gene. The method provided is simple and fast, and has high sensitivity and good repeatability, thus being suitable for wide popularization and application.

4. in the method for detecting an expression level of a CST1 gene in nasal brushing cells provided in the present disclosure, CST1 is a member of cysteine protease inhibitor family, a type 2 cystatin, and abundantly exists in saliva, with a physiological function of inhibiting the destructive action of cysteine proteases such as papain on oral epithelial cells. In recent years, it has been found that CST1 is abnormally expressed in some tumors such as pancreatic cancer and breast cancer, and can exist as a biomarker of aging. The method for detecting an expression level of a CST1 gene in nasal brushing cells provided in the present disclosure can be used for detecting the expression condition of the CST1 in the nasal brushing cells.

5. according to the method for detecting an expression level of a CST1 gene in nasal brushing cells provided in the present disclosure, a relative quantification method of ΔCt or 2^{-ΔΔCt} method is adopted according to actual requirements, the reference gene with relatively constant expression level is selected, the quantity of the reference genes is used for standardization, the target gene expression level is calculated by measuring the difference between the Ct values of the target gene of the sample and the reference gene. The method is simple and fast, with high detection accuracy, may reduce the detection cost, save the detection time, and have advantages such as easy interpretation of result, thus greatly improving the experimental efficiency.

6. the method for detecting an expression level of a CST1 gene in nasal brushing cells provided in the present disclosure provides a foundation for a gene screening technology for detecting the chronic rhinosinusitis with nasal polyps subtype in the future, and provides a reliable basis for clinical guidance and medication, thus ensuring the feasibility of the kit for detecting the chronic rhinosinusitis with nasal polyps subtype in clinical application.

7. the present disclosure proposes for the first time that the Cystatin SN can be used for predicting the sensitivity of the patients with chronic rhinosinusitis with nasal polyps to glucocorticoid, and the detection method is simple and fast, and has good prediction performance, thus having good clinical application prospect.

1. A kit for detecting a subtype of chronic rhinosinusitis with nasal polyps, wherein the kit comprises a specific primer of a CST1 gene.

2. The kit for detecting a subtype of chronic rhinosinusitis with nasal polyps according to claim 1, wherein an upstream primer of the CST1 gene is represented by SEQ ID NO. 2, and a downstream primer of the CST1 gene is represented by SEQ ID NO. 3.

3. The kit for detecting a subtype of chronic rhinosinusitis with nasal polyps according to claim 1, wherein the kit further comprises a specific primer of a reference gene.

4. The kit for detecting a subtype of chronic rhinosinusitis with nasal polyps according to claim 3, wherein the reference gene is GAPDH, wherein an upstream primer of the GAPDH is represented by SEQ ID NO. 4, and a downstream primer of the GAPDH is represented by SEQ ID NO. 5.

5. The kit for detecting a subtype of chronic rhinosinusitis with nasal polyps according to claim 3, wherein the kit further comprises: a reagent for extracting RNA from nasal polyp tissues or from nasal mucosa exfoliated cells; a reagent for making total RNA to undergo reverse transcription to cDNA; and a reagent for performing real-time quantitative PCR reaction on the CST1 gene and the reference gene in the cDNA by adopting quantitative polymerase chain reaction.

6. The kit for detecting a subtype of chronic rhinosinusitis with nasal polyps according to claim 5, wherein the reagent for making total RNA to undergo reverse transcription to cDNA comprises: a reverse transcription mixed solution and RNase-free and DNase-free water; and
the reagent for performing real-time quantitative PCR reaction on the CST1 gene and the reference gene in the cDNA by adopting quantitative polymerase chain reaction comprises:
a PCR premix, double distilled water, a machine fluorescence compensation and correction agent, an upstream primer of the CST1 gene, a downstream primer of the CST1 gene, an upstream primer of the reference gene and a downstream primer of the reference gene.

7. A method for detecting an expression level of a CST1 gene in nasal brushing cells, comprising steps of:
extracting RNA from the nasal brushing cells,
making total RNA to undergo reverse transcription to cDNA,
performing real-time quantitative PCR amplification on the CST1 gene and a reference gene in the cDNA by using a specific primer of the CST1 gene and a specific primer of the reference gene, respectively, by adopting quantitative polymerase chain reaction, and
calculating the expression level of the CST1 gene based on a detection result for an amplification product.

8. The method for detecting an expression level of a CST1 gene in nasal brushing cells according to claim 7, wherein an upstream primer of the CST1 gene is represented by SEQ ID NO. 2, and a downstream primer of the CST1 gene is represented by SEQ ID NO. 3; and
the reference gene is GAPDH, wherein an upstream primer of the reference gene is represented by SEQ ID NO. 4, and a downstream primer of the reference gene is represented by SEQ ID NO. 5.

9. Use of Cystatin SN detection agent in preparation of a kit for predicting, to glucocorticoid, sensitivity of a patient with chronic rhinosinusitis with nasal polyps.

10. The use according to claim 9, wherein the kit further comprises a sample pretreatment reagent.

11. The use according to claim 9, wherein the kit further comprises a reagent for detecting percentage of eosinophils.

12. The use according to claim 9, wherein a dosage form of the glucocorticoid comprises any one of an oral dosage form, an injection, an ointment, a spray and an inhalant.

13. The use according to claim 9, wherein the Cystatin SN detection agent comprises a quantitative detection agent for Cystatin SN protein.

14. The use according to claim 9, wherein the Cystatin SN detection agent is used to detect Cystatin SN mRNA.

15. The use according to claim 14, wherein the Cystatin SN detection agent comprises a reagent suitable for at least one of following methods:
a fluorescent dye method, digital PCR, a resonant light scattering method, real-time quantitative PCR, and sequencing or biomass spectrometry.

16. The use according to claim 14, wherein the Cystatin SN detection agent is a probe or a primer capable of specifically binding Cystatin SN mRNA or Cystatin SN cDNA.

17. The use according to claim 14, wherein the Cystatin SN detection agent is a qRT-PCR primer of Cystatin SN mRNA, of which an upstream primer is represented by SEQ ID NO. 2, and a downstream primer is represented by SEQ ID NO. 3.

18. The use according to claim 14, wherein the kit further comprises a primer of a reference gene.

19. (canceled)

20. (canceled)

21. (canceled)

22. The use according to claim 18, wherein the reference gene is GAPDH, tubulin or actin.

23. The use according to claim 22, wherein a qRT-PCR primer of the GAPDH has an upstream primer represented by SEQ ID NO. 4, and a downstream primer represented by SEQ ID NO. 5.
